(19) United States
(12) Patent Application Publication
(10) Pub. No.: US 2014/0329310 A1
(43) Pub. Date: Nov. 6, 2014

(54) Activated T Cells

(71) Applicant: Lawrence G. Lum, Harrison Township, MI (US)

(72) Inventor: Lawrence G. Lum, Harrison Township, MI (US)

(21) Appl. No.: 14/272,404

(22) Filed: May 7, 2014

Related U.S. Application Data
(63) Continuation of application No. 12/746,581, filed on Sep. 14, 2010, now abandoned.
(60) Provisional application No. 61/005,789, filed on Dec. 7, 2007.

(51) Int. Cl. C12N 5/0783; A61K 35/48

(52) U.S. Cl. CPC C12N 5/0636; A61K 35/51; A61K 2035/124
USPC 435/325

(57) ABSTRACT

Provided are compositions comprising activated T cells and activated T cells armed with bispecific antibodies directed at cancer antigens which are poor responders to alloantigen, and methods of using the compositions to help engraftment and improve anti-tumor effects.

### STATEMENT REGARDING FEDERALLY SPONSORED RESEARCH

This work was partially funded by a Michigan Life Science Grant and NCI R01 CA92344, and the United States government has, therefore, certain rights to the present invention.

### TECHNICAL FIELD

The present invention is directed to activated T cells from cord blood and activated T cells from cord blood armed with bispecific antibodies directed at cancer antigens or to other antigens in bone marrow which are poor responders to alloantigen, and helps hematopoietic engraftment and improves anti-tumor effects.

### BACKGROUND OF THE INVENTION

The treatment of hematologic malignancies with allogeneic stem cell transplant (alloSCT) is limited by numbers of allogeneic donors for alloSCT. Umbilical cord blood stem transplants (CBSCT) has been an important alternative source of hematopoietic stem cells (HSC). However, failure or delayed engraftment remains the critical barrier to the success of CBSCT. The rate limiting step is the absolute number of hematopoietic stem cells (HSC) in the cord blood unit. Despite advances in managing post transplant complications, the limiting factor is HSC dose. Many cord blood (CB) units do not have adequate numbers of HSC to engraft adult patients. Therefore, approaches that increase the effectiveness of a limited number of HSC significantly increase the CB donor pool.

There is a need in the art for enhancing hematopoietic engraftment after CBSCT and the graft-vs-lymphoma effect (GVL) by providing T cell “help” for engraftment and “targeting CD20+” lymphoma with activated T cells (ATC) armed with anti-CD3×anti-CD20 bispecific antibody (CD20Bi). The present invention provides use of targeted ATC from a second cord blood to help engraftment of the cord blood unit used for transplant.

### SUMMARY OF THE INVENTION

The present invention combines properties of cord blood T cells and anti-CD3 activated cord blood T cells (ATC) derived from umbilical cord bloods to be unresponsive to other cord bloods and alloantigens in normal peripheral blood in mixed lymphocyte cultures. Irradiation of the ATC from cord blood decreases the unresponsiveness of the ATC in mixed lymphocyte cultures, indicating the optimization of helper activity and the presence of radiosensitive suppressor or regulatory activity in cord blood T cells or cord blood anti-CD3 activated T cells.

The present invention has industrial applicability and is advantageous in the clinical setting to increase the chances of overcoming graft failure and improving engraftment in patients who receive only a single cord blood transplant or combined cord blood transplants consisting of one or more cord blood products. This approach provides facilitator or helper activity that would be mediated by the cells themselves or products of the cells. This is applicable in all circumstances in patients receiving unrelated stem cell transplants for not only benign and malignant hematologic disorders and solid tumors, but also all benign congenital disorders (e.g. storage disease, sickle cell, thalasemmia, immunodeficiency disorders).

Furthermore, the arming of the cord blood ATC increases the anti-tumor activity directed at residual tumor cells in the body and arming of the cord blood ATC with bispecific antibodies to target bone marrow stem cell niches is expected to provide facilitatory or helper activity that will, again, enhance engraftment of the original or primary cord blood(s) used for engraftment.

### BRIEF DESCRIPTION OF THE DRAWINGS

FIG. 1 shows titration arming dose of cord blood ATC with CD20BI.

FIG. 2 shows expansions of eight cord bloods.

FIG. 3 shows the composition of cord blood ATC.

FIG. 4 shows lack of response of CB and CBATC.

FIG. 5 shows a summary of four cord bloods.

FIG. 6 shows the lack of responses of CB1 to CB2.

FIG. 7 shows the concept in which two mAbs are heteroconjugated and then used to arm ex vivo expanded ATC. The ATC armed with anti-CD3×anti-TAA (Her2=TAA) bind with the TAA on the surface of tumors leading to cytotoxicity and cytokine/chemokine release.

FIGS. 8A and 8B show multiple infusions of ATC in combination with low dose IL-2, and GM-CSF given as immune consolidation after SCT for Stage VI BrCa. FIG. 8B shows overall survival and FIG. 8A shows progression free survival.

FIG. 9 shows HerBi armed ATC vaccination protocol (first part of protocol).

FIG. 10 shows ATC infusions after PBSCT (second part of protocol).

FIG. 11 shows overall survival data in 18 patients.

FIG. 12 shows OKT3 cross-linking to Traut's (1), and Herceptin cross-linking to Sulpho-SMCC (2). The two cross-linked Mabs are allowed to heteroconjugated overnight (3).

FIG. 13 shows the mechanism for cytotoxicity and cytokine release, in which two mAbs are heteroconjugated and then used to armed ex vivo expanded ATC. The ATC armed with anti-CD3×anti-TAA (Her2=TAA) bind with the TAA on the surface of tumors leading to cytotoxicity and cytokine/chemokine release.

FIG. 14 shows that armed T cell production is carried out in a series of steps.

FIG. 15 shows the Stage IV Breast Cancer Protocol used in this Example, in which GM-CSF at a 250 ug/m2/dose and IL-2 at a 300,000 IU/m2/day dose were added as shown

FIG. 16 shows patient, dose, toxicities, and follow-up in Table form.

FIG. 17A shows determining Th1 and Type 1 patterns resulting from taking serum samples at indicated time points over the course of armed. FIG. 17B shows the results from FIG. 17A summarized as a calculation of the mean Th1[IL-2+IFNγ]/Th2 [IL-4+IL-5]=Th1/Th2.

FIG. 18 shows that IFNγ EliSpots were detected during immunotherapy.

FIG. 19 shows persistent cytotoxicity directed at SK-BR-3 in fresh PBMC from patients. PBMC were acquired from whole blood collected at various time points over the course of treatment. All patient PBMC samples were tested for cytotoxic activity against HER2-expressing SK-BR-3 cells (25:1 E/T) and against HER2-negative Raji cells as a negative control.

FIG. 20 shows serum IL-12 levels in patients.

FIG. 21 shows overall survival of Stage IV BrCa. ATC can be expanded ex vivo using anti-CD3 and IL-2 and ATC can be armed ATC.

FIG. 22 shows overall survival of Stage IV BrCa.

FIG. 23 shows heteroconjugation of anti-CD3 and anti-CD20.

FIGS. 24A-C show cytotoxicity mediated by CD20Bi armed ATC from peripheral blood of normals and cancer patients. FIG. 24A shows the results in an ARH-77 target cell line. FIG. 24B shows the results in a B9C target cell line. FIG. 24C shows the results in a Raji target cell line.

FIG. 25 shows anti-CD3 stimulated expansion of CB ATC.

FIG. 26 shows arming doses of anti-CD3×anti-Her2 BiAb

FIG. 27 shows the time course of armed ATC cytotoxicity.

FIG. 28 shows phenotyping on 2 CB after thirteen days.

FIG. 29 shows IFN EliSpots Induced by Her2 or CD20 Binding

FIG. 30 shows specific cytotoxicity mediated by Her2Bi armed ATC as a function of various E:T ratios.

FIG. 31 shows the overall survival of patients treated with Her2Bi-armed ATC.

FIG. 32 shows serum cytokine responses in patients given Her2Bi-armed ATC.

FIG. 33 shows the enhanced specific cytotoxicity mediated by endogenous lymphocytes from patients.

FIG. 34 shows the enhanced specific cytotoxicity that was mediated by endogenous lymphocytes from patients.

FIG. 35 shows that cytotoxicity mediated by CD20Bi-armed ATC is not blocked by free Rituxan®.

FIG. 36 shows that ATC armed with CD20Bi mediate cytotoxicity in Rituxan®-resistant CD20+ ARH-77 cells comparable to cytotoxicity in other CD20+ cell lines, but not in the CD20− cell line, K562.

FIG. 37 shows that CD20Bi-armed ATC secrete cytokines upon binding to tumor antigens.

FIG. 38 shows that an armed ATC from an NHL patient showed significantly enhanced cytotoxicity directed at B9C and ARH-77 compared to unarmed ATC.

FIG. 39 shows the phenotyping of two cord blood samples before and after 13 days of culture.

FIG. 40 shows that ATC derived from Adult Peripheral Blood Mononuclear Cells (PBMC) are allo-non-responsive to alloantigens.

FIG. 41 shows anti-CD20 targeting of mouse CD19 lymphoma cells (A20) in BALB/c mice.

FIG. 42 shows that MNC do not react to Normal Donor (ND) in MLR as strong as ND react to each other.

FIG. 43 shows that ATC can enhance engraftment of umbilical cord blood hematopoietic stem cells (UCBHSC) in SCID/Biege mice.

### DETAILED DESCRIPTION OF THE INVENTION

Applicants' preclinical studies show that peripheral blood T cells can be activated with anti-CD3 mAb, expanded in IL-2, and armed with bispecific antibodies (BiAbs) that target and mediate high levels of specific cytotoxicity against Her2/neu¹⁻³, EGFR⁴, CD20⁵, and CA-125 positive tumors³. In applicants' phase I clinical trials, anti-CD3 activated T cells (ATC) are armed with anti-CD3×anti-Her2/neu bispecific antibody (Her2Bi) for treatment of women with metastatic breast cancer (MBC) and for the treatment of patients with non-Hodgkin's lymphoma (NHL). Arming ATC with Her2B1 or CD20Bi makes every T cell into a specific cytotoxic T lymphocyte (CTL) directed at HER2+ or CD20+ targets^{1;5}. Immune studies in patients who received Her2Bi-armed ATC showed high levels of specific cytotoxicity, elevated serum cytokine levels, decreases in tumor markers, and the suggestion of prolonged overall survival.

In a phase I trial, 15 infusions of ATC armed with CD20Bi were infused after autoSCT for NHL without dose limiting toxicities. In prior studies, applicants showed that ATC could improve engraftment after 9 Gy of TBI in mice transplanted with dose-limiting amounts of HSC⁶. As described in the Examples, cord blood ATC (CBATC) can be expanded, armed with Her2B1 or CD20Bi to mediate high levels of specific cytotoxicity at Her2+ or CD20+ targets, and secrete IFNγ in response to tumor. Furthermore, CBATC and peripheral blood ATC do not respond to alloantigens in mixed lymphocyte culture (MLC) reactions. Therefore, armed CBATC is expected to be a high impact and novel biologic agent for enhancing engraftment and targeting tumors without increasing graft-vs-host disease (GvHD) that can be rapidly translated to the clinic.

Since peripheral blood ATC can suppress autologous peripheral blood MLC responses and CBATC do not respond in alloantigens in the MLC, the invention provides that armed CBATC will enhance engraftment and provide a GVL effect without augmenting GVHD.

Umbilical cord blood transplantation has emerged as a viable option for patients with hematological and non-hematological malignancies that do not have an HLA-matched sibling or matched unrelated donor⁷⁻¹⁰. The advantages of CBT have included rapid access to a donor and a greater tolerance for HLA-disparity due to the naivety of the newborn's immune system. Outcomes with CBT are reported to be comparable to transplant with bone marrow or peripheral blood stem cells with an equal or lesser incidence of acute and chronic GVHD⁷. In contrast, CBT has been limited by a greater incidence in transplant related mortality, primarily due to delays in neutrophil engraftment and immune reconstitution¹¹.

These delays in engraftment and immune reconstitution have resulted not only in increased risk of infectious complication, but also relapse. This risk of relapse is further exacerbated by the relatively naïve population of T cells in the cord blood unit. In adults, the delay in immune reconstitution has been attributed to decreased thymopoiesis leading to a reduction in the number and function of lymphocytes¹². Thus, strategies to hasten engraftment and immune reconstitution and decrease relapse rates are expected to lead to improved outcomes following CBT.

Enhancing Engraftment and Immune Reconstitution.

The number of total nucleated and CD34+ cells have been identified as the most fundamental parameters that portend engraftment and outcomes following CBT¹³. To date, CBT has been most successfully employed in children, but has been limited in adults by insufficient cell numbers in a single cord blood unit¹⁴. Strategies that have been employed in an effort to circumvent the limitation of cell dose have included the co-transplantation of more than one cord blood unit and the ex vivo expansion of a single cord blood unit^{10;13}. While transplantation with ex vivo expanded cord blood cells have failed to enhance engraftment in clinical studies, both pre-clinical and clinical studies have shown that the co-transplantation of two or more cord blood units significantly improves the rate of engraftment over a single cord blood transplant when an insufficient cell dose is administered^{15 10;13}. Furthermore, patients receiving double cord blood transplants have similar rates of acute and chronic GVHD, transplant-related mortality, and disease-free and overall survival when similar cell doses are compared between single and double cord blood transplants¹⁶. These findings support umbilical cord blood as a worthy stem cell source for alloSCT and warrant further investigation into methods of optimization of engraftment and immune reconstitution.

Furthermore, several lines of evidence suggest that the co-infusion of a second cord blood may contain “facilitator cells” that serve to enhance engraftment of the other cord blood unit, while long term hematopoiesis is generally derived from a single cord blood unit^{8;13}. The hypothesis that one cord blood unit facilitates the reconstitution of the other is supported by studies showing that the co-infusion of peripheral blood stem cells from another donor enhances the long term engraftment of the cord blood cells. In this case, peripheral blood stem cells were engrafted initially at day 10 followed by full cord blood engraftment by day 22 post transplant¹⁷. Without being bound by a specific mechanism, it is relevant that Takura et al¹⁶ reported that the CD8+ cell dose improved engraftment following CBT in patients with a suboptimal CD34+ cell dose, suggesting that a subset of T cells may be the requisite facilitator cells.

Indeed, in applicants' earlier study¹⁸, applicants showed that anti-CD3 activated murine splenocytes (ASC) were able to enhance survival in lethally irradiated (9 Gy) 6-8 weeks old female BDF1 (C57BL6×DBA, H2^{b}/H2^{d}) mice after transplant of syngeneic HSC containing splenocytes. There was a significant enhancement in OS after transplant in mice given dose-limiting numbers of unmanipulated splenocytes (SC). The infused ASC population consisted of 77.6% CD3+ cells; however, the particular T cell subpopulation responsible for the enhanced survival was not determined. More recently, Hexner et al¹⁹ showed that T cells can enhance hematopoietic engraftment beyond overcoming immune barriers by stimulating stem cell differentiation in human xenografts in immunodeficient mice. Consistent with these findings, immune reconstitution is significantly delayed following T-cell depleted alloSCT suggesting that this cell population plays an important role. Thus, defining and characterizing facilitator cell population(s) would provide an opportunity to enhance engraftment and immune reconstitution in CB units when cell number is dose-limiting.

### Summary of CBATC Expansion, Specific Cytotoxicity, and Decreased Alloreactivity.

CBATC can be consistently expanded in IL-2 from cryopreserved CB units nearly two logs in 2 weeks. Consistent with applicants' findings using peripheral blood, CBATC can be armed with Her2B1 or CD20Bi to target Her2+ and CD20+ targets. Dose titrations show that the optimal arming doses are 50 ng of each BiAb/10⁶ ATC. Specific cytotoxicity as measured by ⁵¹Cr release assays or by IFNγ Elispots peaked between 8 and 12 days for both BiAbs. The ability of CD20Bi armed ATC to produce EliSpots for IFNγ were tested by arming ATC after 0, 8, 11, and 13 days of culture with CD20BI and targeting CD20+ targets and SK-BR-3 (irrelevant targets) by mixing the armed ATC with each target. Only CD20+ targets induced EliSpots.

These studies show that functional, specific, and cytotoxic T cells can be generated from fresh or frozen CB and may be used to target tumors cells in vivo. The MLC data strongly support the use of CBATC derived from a second CB unit to help the primary CB unit engraft. Furthermore, the non-responsiveness between cord bloods supports the possibility that the use of a second armed or unarmed CBATC in combination with a first or primary CBSCT is not only feasible but may lead to improve engraftment and decreased alloreactivity (e.g., GVHD).

Enhancing Graft Vs. Lymphoma.

GVL following alloSCT plays a major role in eradicating residual disease following chemotherapy with both myeloablative and nonmyeloablative conditioning regimens²⁰. However, relapse and GvHD are major limitations to success in patients with resistant or refractory NHL. Although GvHD provides the benefit of overlapping GVL effect, it is restricted by its toxicity and high mortality rate. In an effort to maximize GVL and minimize GvHD, donor leukocyte infusions (DLI) have been used and shown to induce remissions in hematological malignancies²¹. However, GVHD induced by the DLI remains a serious barrier to the overall success of alloSCT. Therefore, the infusion of targeted T cells may augment GVL while minimizing GVHD and targeted CB T cells may provide an excellent source of T cells to further enhance a GVL effect.

Use of Activated T Cells (ATC) and AutoSCT for Stage IV Breast Cancer.

Cross linking T cell receptors with anti-CD3 mAb leads to T cell activation, proliferation, cytokine synthesis, and non-specific cytotoxicity directed at tumor targets. These ATC can be expanded in IL-2 (100 IU/ml), exhibit non-MHC restricted cytotoxicity, and produce IFNγ, TNFα and GM-CSF²²⁻³¹. In animal models, infusions of ATC provided anti-tumor effects. Murine studies suggested that infusing anti-CD3-activated CD4 cells during the nadir after cyclophosphamide in combination with IL-2 may induce remissions more effectively. Applicants have shown in applicants' phase I trial that immunotherapy consolidation using multiple infusions of ATC with low dose IL-2 and GM-CSF may provide an anti-breast cancer tumor effect after HDC and PBSCT. No ATC dose-limiting toxicities (DLT) were observed. OS and PFS were 70% and 50%, respectively at 32 months whereas OS and PFS for patients who underwent autoSCT alone was 50% and 15%, respectively³². Not only can polyclonal ATC provide anti-tumor activity after autoSCT, applicants' subsequent studies were undertaken to determine if ATC armed with BiAb provide even greater anti-tumor activity with increased specificity to tumor associated antigens.

Targeting Tumor-Associated Antigens (TAA) Using BiAbs.

Adoptive immunotherapy of antigen-specific T cells has been successfully employed in both pre-clinical and clinical studies in applicants' laboratory as well as others. Targeting ATC to tumor-specific antigens can be achieved by heteroconjugating an anti-CD3 mAb with a mAb against a tumor associated antigen (anti-TAA) as depicted in the cartoon below. Applicants have used this strategy to demonstrate that ATC can be armed with anti-CD3×anti-HER2, a member of the epidermal growth factor receptor family of tyrosine kinases that is overexpressed in a variety of cancers. Her2 Bi-armed ATC have been shown to exhibit cytotoxic activity toward the Her2/neu-expressing breast cancer cell line SK-BR-3¹. Specific cytotoxicity directed toward PC-3 prostate adenocarcinoma cells have also been observed with Her2 Bi armed ATC both in vitro and in a Beige/SCID mouse model³³. Preliminary results from an ongoing phase I dose escalation clinical trial is being done in women with stable to progressive HER2-positive or HER2-negative metastatic breast cancer to determine the maximum tolerated dose (MTD) for Her2Bi-armed ATC given with IL-2 and GM-CSF. Results have been encouraging with no dose limiting toxicities observed to date and an increase in overall survival in the HER2 positive group. Activated T cells armed with BiAbs directed against the EGFR receptor have also been shown to induce killing of cancer cells derived from a variety of tissue sources, including lung, pancreas, colon, brain, and skin⁴. Furthermore, EGFRBi-armed ATC inhibited the growth of COLO356/FG pancreatic and LS174T colorectal tumors in the Beige/SCID mouse model. Using a similar strategy, cytokine induced killer cells (CIK) that have been expanded and armed with BiAbs have been shown to enhance tumor killing. CIKs obtained from patients with ovarian cancer armed with antibodies directed against CA-125 and Her2 exhibited enhanced killing of primary ovarian cancer cells³⁴. Cytotoxicity toward CD19 positive B cells was also observed with CIK armed with BiAbs toward CD19 (CD19×CD5)³⁵. These studies demonstrate that ATC armed with BiAbs are effective at tumor killing both in vitro and in vivo.

Targeting the CD20 Antigen on B Cell Malignancies.

CD20 is a 33-36 kDa non-glycosylated phosphoprotein expressed on the mature B cells except for plasma cells. CD20 expression on precursor B-cells in the bone marrow occurs simultaneously with downregulation of CD34 expression. Since CD20 is expressed by >95% of B cell NHLs and other B-cell malignancies but is absent on B-cell precursors, dendritic cells, and plasma cells, it is an attractive target to induce tumor specific killing. Rituxan®, a chimeric mAb directed at CD20 expressed by B cells, was the first mAb approved for cancer treatment. Rituxan® induces killing of CD20+ cells both directly, via complement-mediated cytotoxicity (CMC) and antibody-dependent cell-mediated cytotoxicity (ADCC), and indirectly via induction of apoptosis and sensitization to chemotherapy. Rituxan® is well-tolerated with only mild (Grade I/II) toxicities associated with tumor-lysis syndrome, in the presence of bulky disease, or cytokine-release syndrome. Treatment of relapsed or refractory follicular lymphoma with Rituxan® results in remissions in ˜50% of patients, with durations of about 1 year^{36;37}. Complete remissions induced by Rituxan® have been reported as high as 26% in patients with low tumor burdens³⁸ but are rare (only ˜5%) in patients with greater tumor burdens³⁹. Response rates to Rituxan® for other B-cell NHL is also significantly lower (˜10-15%)³⁶. Only 40% of patients who initially respond to Rituxan® will respond after relapse. Therefore, Rituxan-resistance presents a major limitation to successful therapy; combining Rituxan® with other cytotoxic or biologic agents may improve outcome. Phase II studies exploring Rituxan® combined with CHOP showed significant improvements with a 95% response rate and complete remissions in 55% of patients. However, randomized studies are needed to determine the benefits of combined therapy with Rituxan® for preventing relapse and improving overall survival.

Adoptive Immunotherapy Using CD20Bi Armed ATC to Treat NHL.

ATC armed with anti-CD3×anti-CD20 (Rituxan®) BiAb (CD20Bi) are effective in lysing Rituxan®-resistant CD20+ B cells⁴⁰. Arming ATC with CD20 Bi enhanced cell lysis of three CD20+ cell lines, including B9C, Raji, ARH-77, but not the CD20− K562 cells compared to unarmed ATC and ATC armed with irrelevant Her2Bi⁴⁰. Since applicants' data suggested that CD20Bi armed ATC may be clinically effective for Rituxan®-resistant CD20+ hematological malignancies, Applicants initiated a phase I trial using infusions of CD20Bi-armed ATC for patients with NHL following HDC and PBSCT. Three patients have been enrolled at the first dose level of 5×10⁹ CD20Bi-armed ATC per infusion (75×10⁹ total dose of armed ATC) and one patient enrolled at the 10×10⁹ dose level (total of 15×10¹⁰). All are alive and 2 patients are in remission with 1 in remission after a reduced intensity alloSCT for relapse.

Immunotherapy Using Cord Blood Cells.

CB cells are an alternative stem cell source for patients, who lack a related or unrelated HLA-matched donor for alloSCT^{8;9}. As mentioned earlier, these cells offer the unique opportunity to optimize the GVL effect while minimizing GVHD. Applicants hypothesize that CBATC can be expanded ex vivo, armed with BiAbs to target tumor targets while providing help for engraftment. Indeed, applicants' preliminary data show that CBATC can be produced by stimulation anti-CD3 and expanded in IL-2 using the same well described methods and SOPS^{33;41}. CBATC derived from cord blood units exhibit specific cytotoxicity directed toward CD20+ and Her2/neu targets in vitro (see Examples 17-25) when armed with CD20Bi and Her2Bi, respectively. By infusing armed CBATC after HDC and CBSCT, it is likely that the specific CTL activity would be enhanced. Recently, Wrzesinski et al⁴² showed that lymphodepletion preferentially induces the expansion of adoptively transferred CD8+ cells, while Rapaport et al⁴³ reported that T cell infusions given after HDC and autoSCT resulted in significantly improved T cell proliferation. Thus, lymphodepletion may enhance the expansion of armed ATC after HDC and provide a GVL effect to decrease relapse. In contrast, armed CBATC do not proliferate in response to alloantigens in MLC reactions suggesting the benefit of an enhanced GVL without augmenting GvHD. Multiple armed CBATC infusions will be used to enhance engraftment without increasing GvHD in a phase I/II protocol using armed CBATC to treat patients with high risk CD20+ hematologic malignancies after HDC and cord blood stem cell transplant (CBSCT). The feasibility of this approach is supported by applicants' pre-clinical and ongoing phase I/II clinical studies involving armed ATC infusions directed at Her2/neu in MBC patients and at CD20+ in NHL patients after autoSCT. Studies by others also support this approach. Wei et al reported that T, NK, and CD34+ cells could also be expanded from cord blood⁴⁴ and Kobari et al⁴⁵ showed that the ex vivo expansion of CB CD34+ cells did not alter the capacity to generate functional T lymphocytes or dendritic cells. CB T cells have also been expanded and engineered with chimeric receptors targeting CD19 to improve the anti-tumor properties by other investigators. Serrano et al⁴⁶ demonstrated that CB T cells could be genetically modified to express a chimeric CD19 receptor that targets and kills CD19 tumor targets in vitro and reduced the size of CD19+ tumors in vivo in NOD/SCID mice. Furthermore, when CB derived ex vivo expanded T cells expressing the chimeric CD19+ receptor were co-infused with an anti-CD20-IL2 immunocytokine, CD19+CD20+ tumors were reduced to a level beyond that achieved with either treatment alone⁴⁷. These studies demonstrate that CBATC can be expanded ex vivo, armed with CD20Bi to exhibit antigen-specific cytotoxicity both in vitro and in vivo and be used for the development of strategies to enhance GVL while minimizing GVHD following CBSCT.

It should be understood that the Examples and embodiments described herein are for illustrative purposes only and that various modifications or changes in light thereof will be suggested to persons skilled in the art and are to be included within the spirit and purview of this application.

### EXAMPLES

### Example 1

### Cord Blood Graft Enhancement and Targeting

Cord blood is an excellent source of stem cells. This Example describes the observation that cord blood helps each other engraft in double cord blood transplant since the stem cell dose from one is often not enough in adults. More patients can receive stem cell transplants if donor availability were increased and if engraftment can be enhanced Earlier data show that ATC enhance engraftment occurs in mice that are lethally irradiated. These data show that ATC expanded from splenocytes can provide helper activity to engraftment in a murine system.

This Example was performed to determine if ATC can be grown that enhance engraftment and be targeted to kill lymphoma cells. CBATC can be expanded for clinical use. CBATC can be armed with targeting bispecific antibodies to kill solid tumors and lymphomas (CD20+). CB Mononuclear cells do not react to alloantigens. CBATC do not react to alloantigens. Different CB or CBATC do not react to each other.

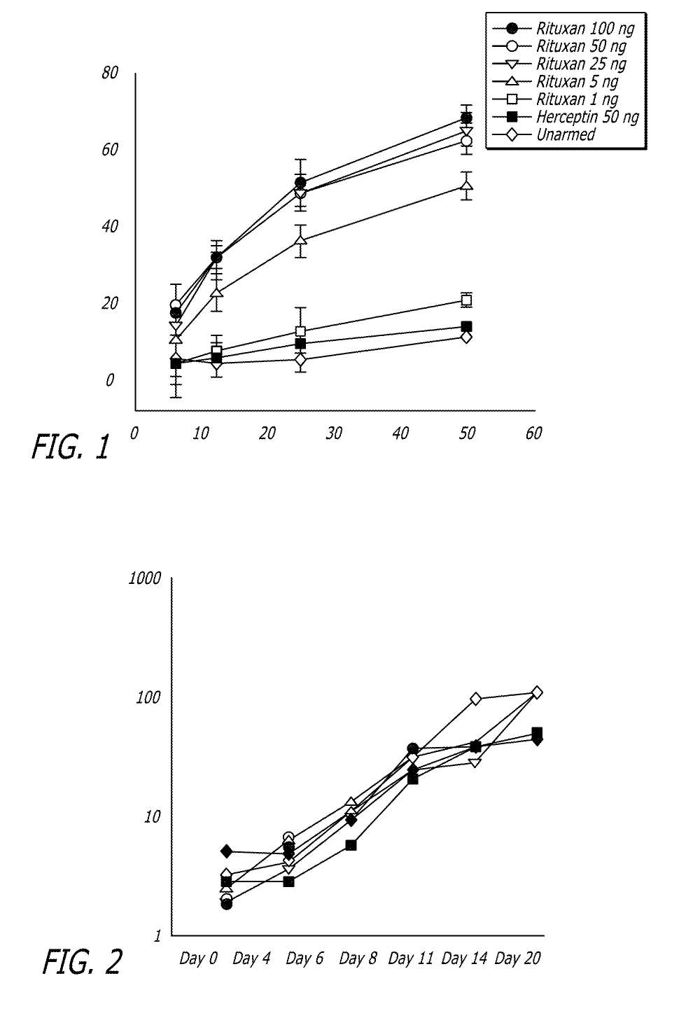

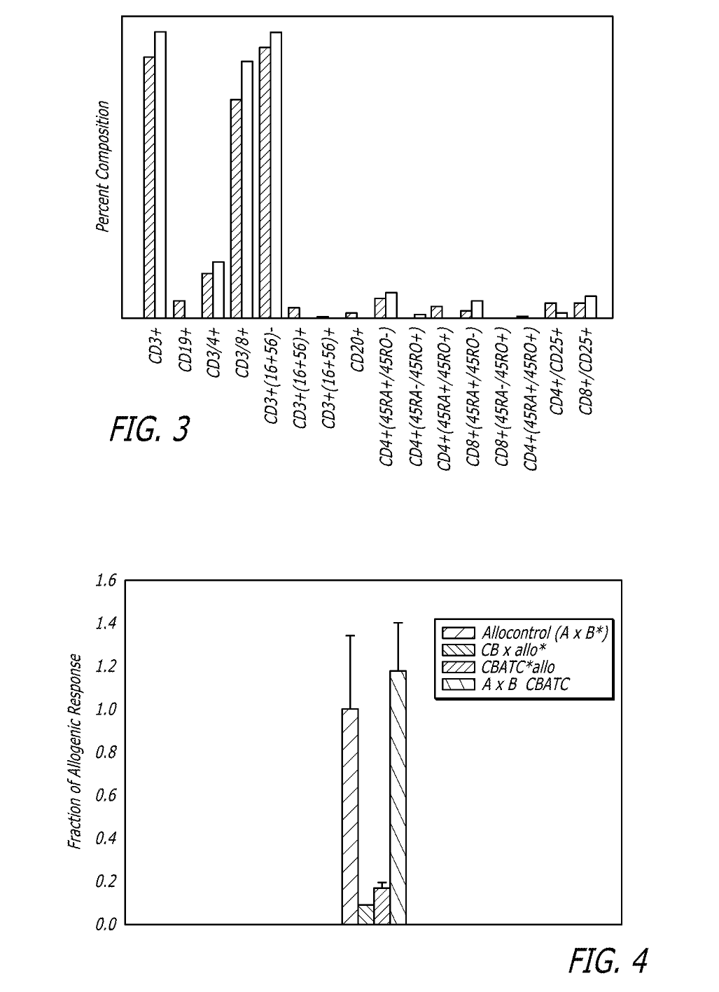

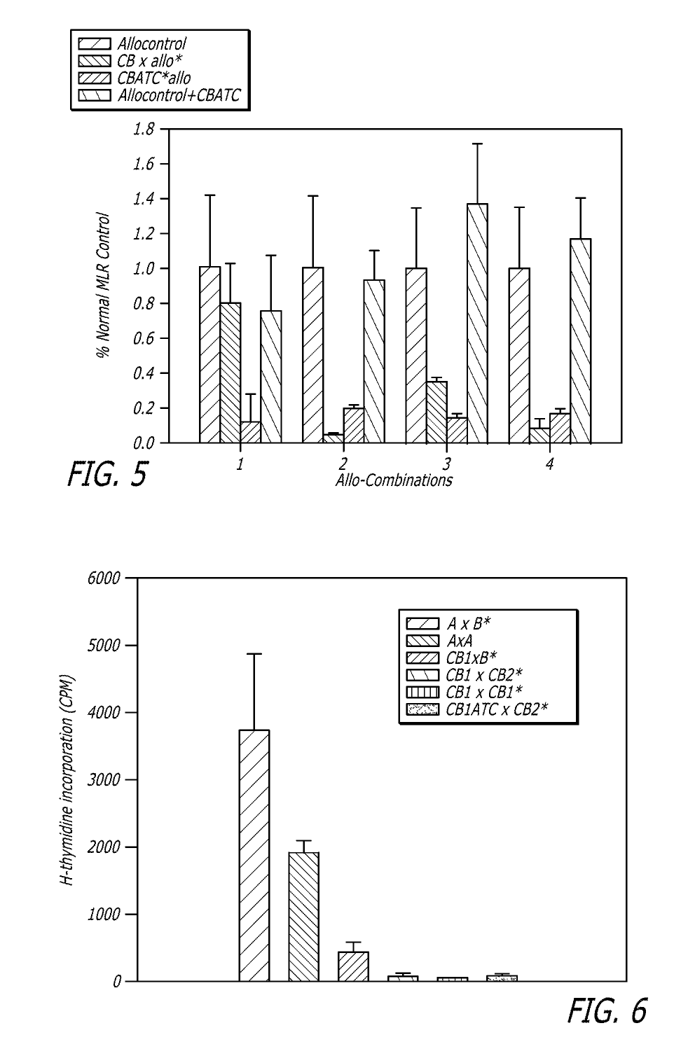

FIG. 1 shows titration arming dose of cord blood ATC with CD20BI. FIG. 2 shows expansions of eight cord bloods. FIG. 3 shows the composition of cord blood ATC. FIG. 4 shows lack of response of CB and CBATC. FIG. 5 shows a summary of four cord bloods. FIG. 6 shows the lack of responses of CB1 to CB2. In summary, therefore, CB can be used together to boost engraftment or enhance anti-tumor killing after cord blood transplants.

### Example 2

### Anti-CD3 Activated T Cells from Cord Blood can be Expanded Ex Vivo and Armed with Bispecific Antibodies to Lyse HER2/Neu+ and CD20+ Targets

Applicants' previous studies showed that anti-CD3 activated T cells (ATC) from peripheral blood mononuclear cells could be expanded in interleukin-2 (IL-2) for 14 days and armed with anti-CD3×anti-Her2/neu (Her2Bi) [J Hemat and Stem Cell Res 10:247, 2001], anti-CD3×anti-CD20 (CD20Bi) [Exp Hemat 33:452, 2005], or anti-CD3×anti-EGFR [Clin Cancer Res 12:183, 2006] bispecific antibody (BiAb) and can kill Her2/neu, CD20, and EGFR+ tumor targets, respectively. This Example was performed to determine whether anti-CD3 activated cord blood T cells (CBATC) could be expanded and targeted with Her2B1 and CD20Bi to tumors or hematologic malignancies for infusions after cord blood stem cell transplant (CBSCT).

CB mononuclear cells were activated with anti-CD3 (20 ng/ml) and expanded for 14 days in IL-2 (100 IU/ml). CBATC were armed with Her2B1 or CD20Bi and tested for specific cytotoxicity directed SK-BR-3, Raji, or B9C targets and cytokine secretion or IFNγ EliSpots after binding to tumor cells. The results show the mean expansion of CBATC to be 43-fold (n=8) after 14 days of culture. By the end of culture, the proportions of CD8+ and CD4+ were 82% and 18%, respectively. The proportion of cells expressing CD19 or CD20 did not exceed 6.3%, CD56+ cells were <3.6% and CD3−CD16+CD56+ cells was <0.7%. Cells positive for CD4+CD25+ or CD8+CD25+ were 4.2% or 7.1%, respectively (n=2). Specific cytotoxicity was optimized when CBATC were armed with 50 ng/10⁶ cells of Her2B1 or CD20Bi (arming dose ranged from 5, 50, and 500 ng/10⁶ cells); arming significantly increased cytotoxicity of the armed CBATC over that seen for unarmed CBATC. Cytotoxicity peaked between days 12 and 14 for both BiAbs.

The ability of CD20Bi armed ATC to produce Elispots for IFNγ was tested by arming ATC the CD20Bi after 0, 8, 11, and 13 days of culture peaked on day 8. Only CD20+ targets induced Elispots and day 8 armed ATC exhibited peak numbers (2,200 Elispots (ranged from 1,700 to 1,800 on day 8)/10⁶ armed ATC plated). At an effector/target ratio (E:T) of 25:1, the mean cytotoxicity of CBATC armed with Her2B1 or CD20Bi was 60% (n=4) and 35% (n=1), respectively. In an extended culture to day 47, mean cytotoxicity for Her2Bi-armed CBATC was 36% at an E/T of 25:1 compared to 4.35% for unarmed CBATC. Unarmed CBATC did not kill Daudi targets. Armed CBATC mediated both specific cytotoxicity and secreted IFN-γ as measured by ELISA or EliSpots. Both fresh and frozen CB could be used in the assays. In a clinical application, specific cytotoxicity of armed CBATC could be used to augment anti-tumor and anti-lymphoma effects after CBSCT.

### Example 3

### Enhanced Anti-Breast Cancer Cytotoxicity after Autologous Peripheral Blood Stem Cell Transplant (PBSCT) by Boosting with Multiple Infusions of Activated T Cells (ATC) Armed with Anti-CD3×Anti-HER2 Bispecific Antibody (HER2Bi) Prior to PBSCT

More aggressive treatment strategies are needed for women with metastatic breast cancer (mBrCa). Although peripheral blood stem cell transplant (PBSCT) permits the use of high-dose chemotherapy (HDC) that could not otherwise be given, results for PBSCT have not been encouraging. In order to boost tumor kill, applicants combined a protocol that targets Her2 using activated T cells (ATC) armed with anti-CD3×anti-Her2 bispecific antibody (Her2Bi) with a second protocol that involves infusions of ATC after PBSCT to boost anti-tumor immunity.

In applicants' phase I trial using ATC with Her2Bi to treat women with mBrCA who have Her2 positive or negative disease, applicants found that multiple infusions of armed ATC induced cytotoxicity directed at BrCa cells in the peripheral blood mononuclear cells (PBMC) of the patients that develop in 2 weeks and last up to 4 mos (Clin Canc Res 12:569, 2006). Armed ATC lyse tumors that are Her2 low-expressors (0-1+). Targeting leads to specific cytotoxicity, induces cytokine/chemokine release, and proliferation of the armed ATC. In a second study, ATC were infused after PBSCT into 23 women with mBrCa leading to 70% overall survival and 50% progression free survival at 32 mos after PBSCT.

Therefore, the combined strategy involved obtaining T cells by another leukopheresis after the boosting with armed ATC, expanding the immune T cells and infusing the expanded ATC after PBSCT to transfer pre-immune anti-BrCa cytotoxicity to reconstitute cytotoxicity after PBSCT. Two patients (one pt was Her2 3+; one pt was Her2 0-1+) underwent treatment with this treatment approach. Both patients were given 8 infusions (20 billion/infusion with a total of 160 billion) of ATC armed with Her2Bi in the first protocol and subsequently leukopheresed and ATC were produced for the second protocol.

The expanded ATC at an effector:target ratio (E/T) of 25:1, exhibited cytotoxicity at BrCa tumor cells (SK-BR-3) at 71% and 75% for pts 1 and 2, respectively. The cell product for pt 1 contained 68% CD3+, 32% CD4+, 39% CD8+, 29% CD16+56+, 12% CD4+CD25+, and 15% CD8+CD25+ cells and the cell product for pt 2 contained 35% CD3+, 25% CD4+, 8.5% CD8+, 23.3% CD16+56+, 10.4% CD4+CD25+, and 4.7% CD8+CD25+ cells. The protocol involves infusing 15 doses of ATC after PBSCT with 3 doses of ATC/week for 3 weeks and then ATC once/week for six more weeks. The pt1 and pt 2 received total of 114 and 70 billion ATC, respectively. Pt1 developed anti-BrCA cytotoxicity of 9% 2 weeks after PBSCT. Pt2 exhibited anti-BrCa cytotoxicity at an E:T of 25:1 of 38% and 15% at 3 weeks and 6 months after PBSCT, respectively.

Phenotyping of peripheral blood at 6 mos after PBSCT showed 61% CD3+, 36% CD4+, 19.5% CD8+, and 15.5% CD56+ cells. There was no cytotoxicity directed at Daudi cells. These data strongly suggest that transfer of pre-immune cells after PBSCT accelerates immune reconstitution of tumor specific cytotoxicity after PBSCT. The preboost strategy with targeted T cells is being combined in a proof of principle trial to assess whether enhanced cytotoxicity can be consistently enhanced after PBSCT.

### Example 4

### Induction of Immune Responses and Improved Survival after Infusions of T Cells Armed with Anti-Cd3 X Anti-HER2/Neu Bispecific Antibody in Stage IV Breast Cancer Patients (Phase I)

Women with stage IV metastatic breast cancer (mBrCa) have limited treatment options since toxicities from chemotherapy and radiotherapy become limiting. Non-toxic immunotherapy approaches to improve targeting and cytotoxicity directed at breast cancer are needed. Applicants' earlier study showed that anti-CD3 activated T cells (ATC) could be expanded in culture and then armed with anti-CD3×anti-Her2/neu bispecific antibody (HER2Bi). The armed ATC mediate enhanced specific cytotoxicity, proliferate, and induce cytokine/chemokine secretion (J Hematotherapy and Stem Cell Res 10:247, 2001).

In a Phase I trial using ATC armed with Her2Bi, 18 Stage IV BrCa patients (pts) were treated with 8 infusions (twice/week) for 4 weeks totaling 40 (6 pts), 80 (2 pts), 160 (7 pts), and 320(1 pt) billion ATC armed with Her2Bi without dose-limiting toxicities. The most frequent side-effects were chills, fever, and hypotension that were easily controlled with medications. Two stage IV mBrCa patients had minor responses with decreases in CEA (35.2 to 4.1 ng/ml) or CA 27-29 (57.7 to 35.6 U/ml) and one pt had partial response with a decreased liver metastatic lesion. None of the pts developed human anti-mouse antibodies levels above 10 ng/ml. Immunoaffinity-depletion of BiAb-armed ATC from PBMC of a high risk IV mBrCa pt at post-treatment time points showed an increase in anti-BrCa tumor cell activity exhibited by endogenous immune cells that persisted up to 4 months after treatment.

Increasing proportions and absolute numbers of CD25+ cells in CD4+ and CD8+ subsets were observed as a function of treatment with nearly all CD4+ and CD8+ cells being CD25+ by 1 week post-final infusion. Significant treatment-associated elevations (several log increases over baseline) of circulating IFNγ, TNFα, IL-2, IL-5, IL-10, IL-12p70, and IL-13 were detected in serum of nearly all of the patients beginning 1-2 weeks after initiation of infusions. Particularly remarkable was the 3 log increase of mean (n=9) serum IL-12p70 from 0 to 1200 pg/ml. There was a Th1 shift that persisted during therapy.

To date, results from the phase I clinical trial suggest that Her2Bi-armed ATC activate the endogeneous immune system to generate an adaptive immune responses despite the presence of high tumor burdens. FIG. 22 shows the overall survival for 18 women (All) treated on the phase I protocol with the median survival not yet defined for the HER2/neu 3+ group and the entire study group. The median survival for the 9 pts with Her2/neu negative disease was 21.5 months. Together these data are encouraging and strongly suggest infusions of armed targeted T cells may immunized the patient against their own tumor antigens leading to immunoreactivity manifested as the development of a persistent CTL response that may lead to improved overall survival.

### Example 5

### Use of Anti-CD3 Activated T Cells (ATC) from Women with Stage IV Metastatic Breast Cancer

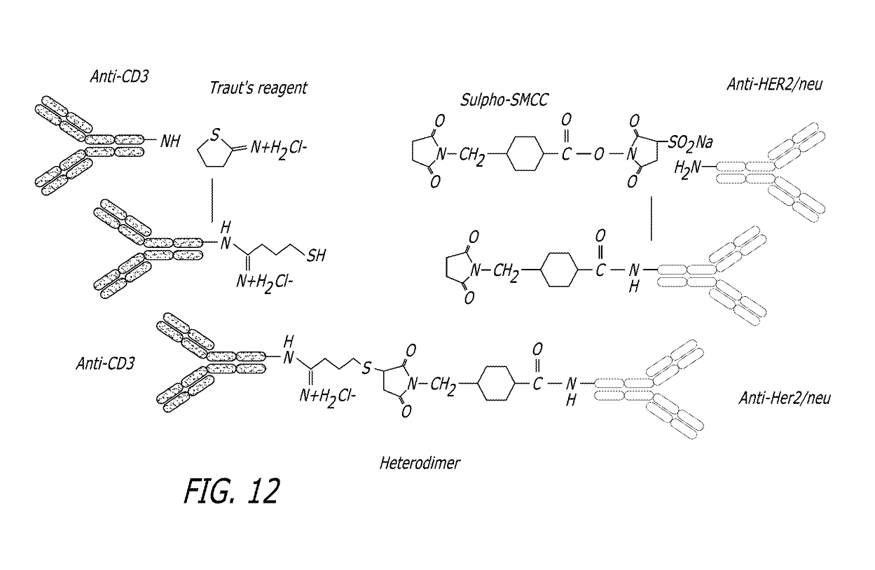

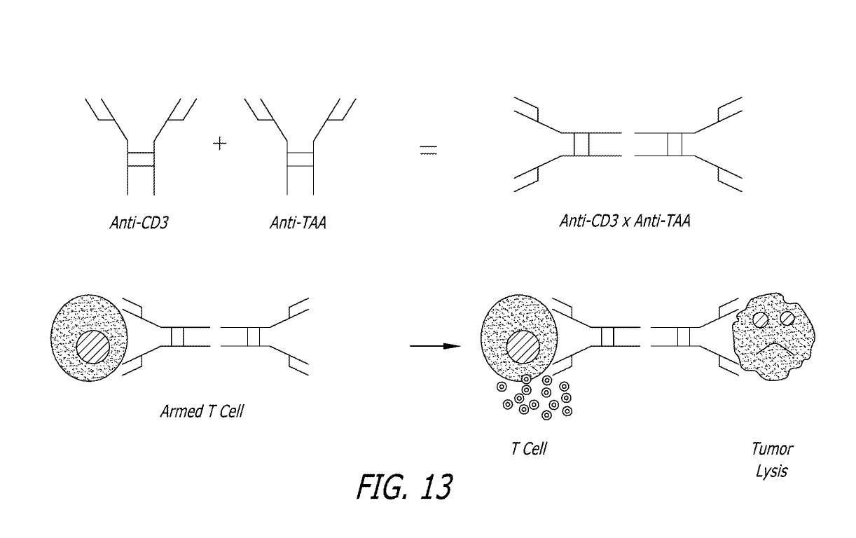

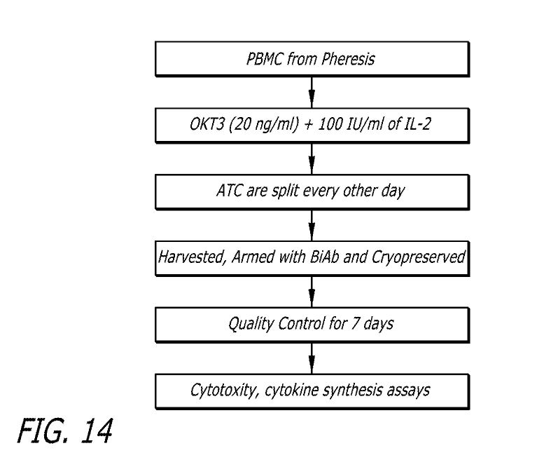

The primary aim of this Example was to determine whether anti-CD3 activated T cells (ATC) from women with stage IV metastatic breast cancer could be expanded and targeted to Her2/neu in a phase I dose escalation trial of Her2Bi armed ATC. The secondary aims of this Example included evaluating clinical responses and immune correlates. The methods used herein follow from the methods used in Example 6. FIG. 12 shows OKT3 cross-linking to Traut's (1), and Herceptin cross-linking to Sulpho-SMCC (2). The two cross-linked Mabs are allowed to heteroconjugated overnight (3). FIG. 13 shows the mechanism for cytotoxicity and cytokine release, in which two mAbs are heteroconjugated and then used to armed ex vivo expanded ATC. The ATC armed with anti-CD3×anti-TAA (Her2=TAA) bind with the TAA on the surface of tumors leading to cytotoxicity and cytokine/chemokine release. As shown in FIG. 14, armed T cell production is carried out in a series of steps.

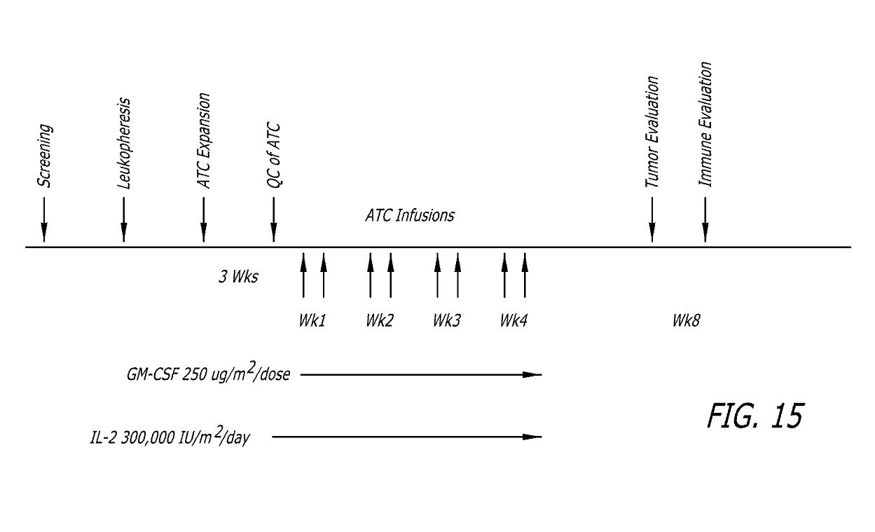

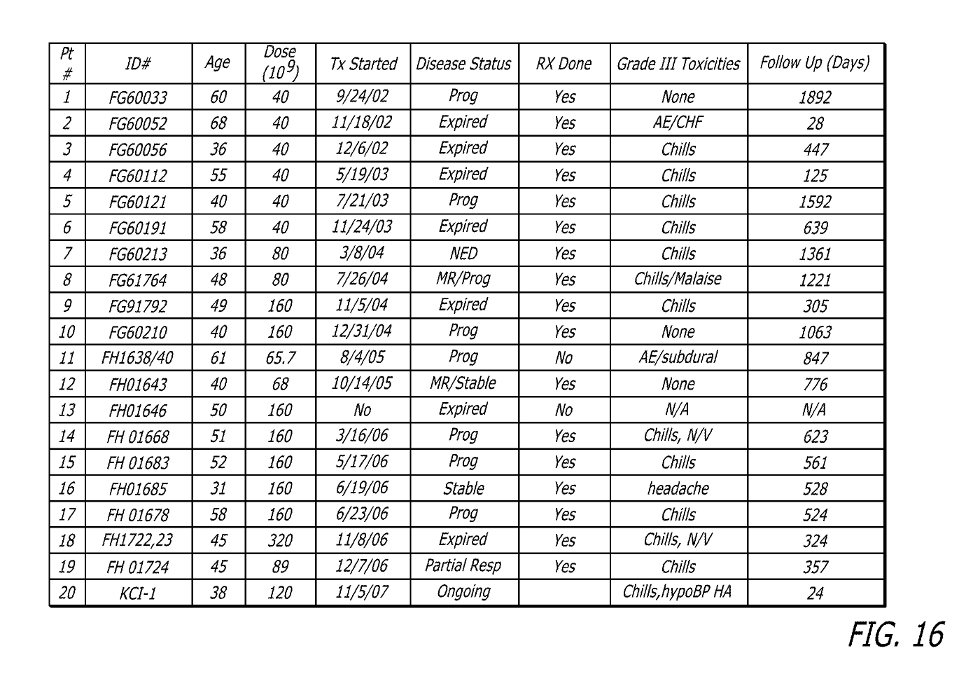

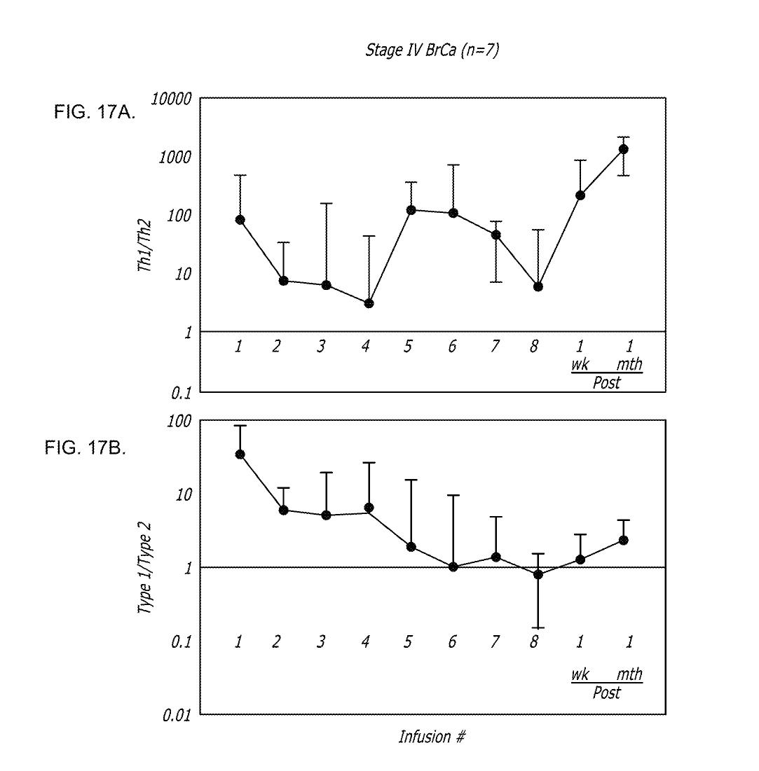

FIG. 15 shows the Stage IV Breast Cancer Protocol used in this Example, in which GM-CSF at a 250 ug/m2/dose and IL-2 at a 300,000 IU/m2/day dose were added as shown. Patient, dose, toxicities, and follow-up are shown in Table form in FIG. 16. To determine Th1 and Type 1 patterns, serum samples, taken at indicated time points over the course of armed ATC infusions, were tested for cytokines (IL-2, IL-4, IL-5, IL-10, IL-12 p70, IL-13, GM-CSF, IFN, and TNF-α) representative of Th1- and Th2-type immune responses, as shown in FIG. 17A. These results are summarized as a calculation of the mean Th1[IL-2+IFNγ]/Th2 [IL-4+IL-5]=Th1/Th2. The results show a Th1 as function of armed ATC infusions. Overall immune response, calculated as the average ratio of Type 1 [IL-2+IFNγ]/Type 2[IL-4+IL-5+IL-10+IL-13], remained polarized towards a Th1-type response throughout treatment, as shown in FIG. 17B.

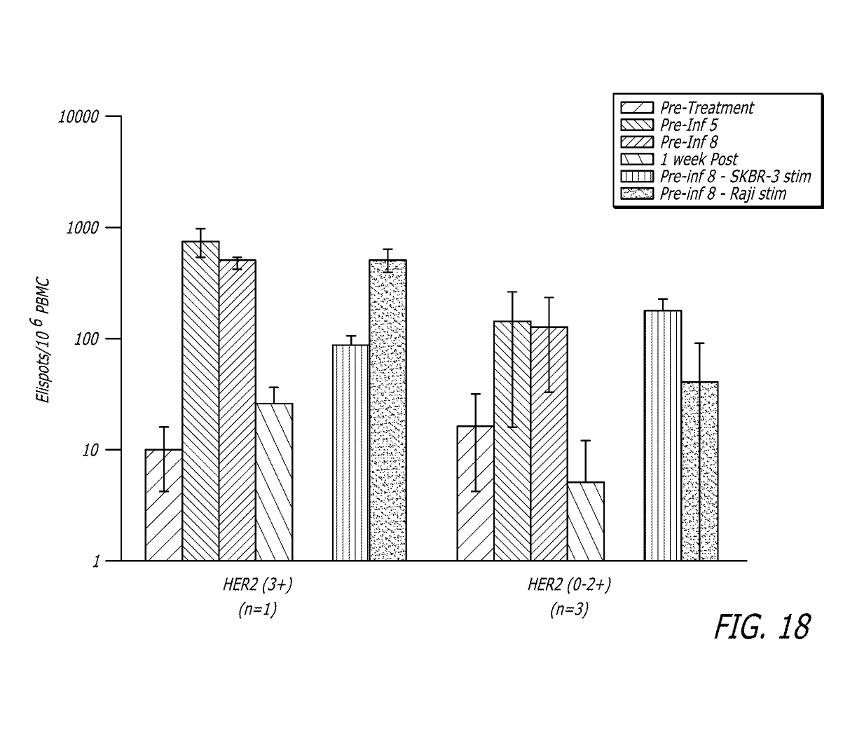

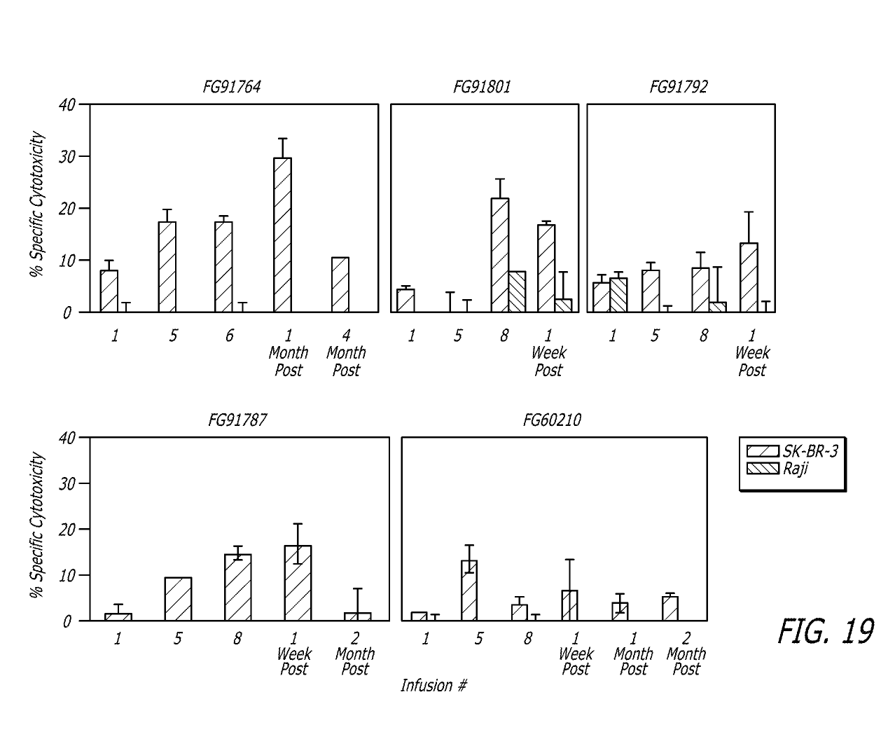

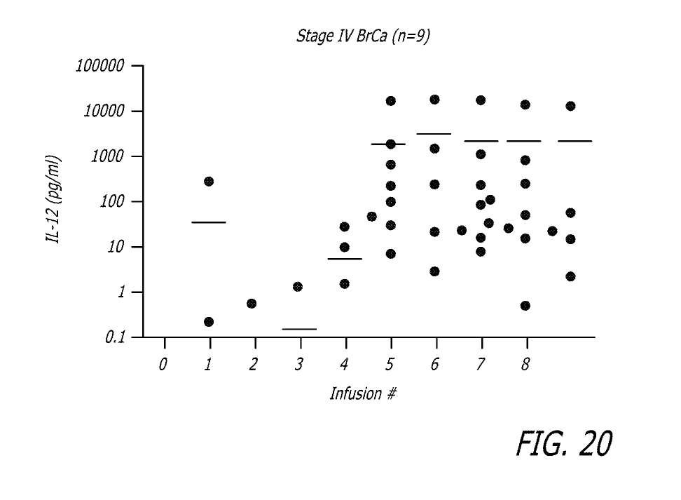

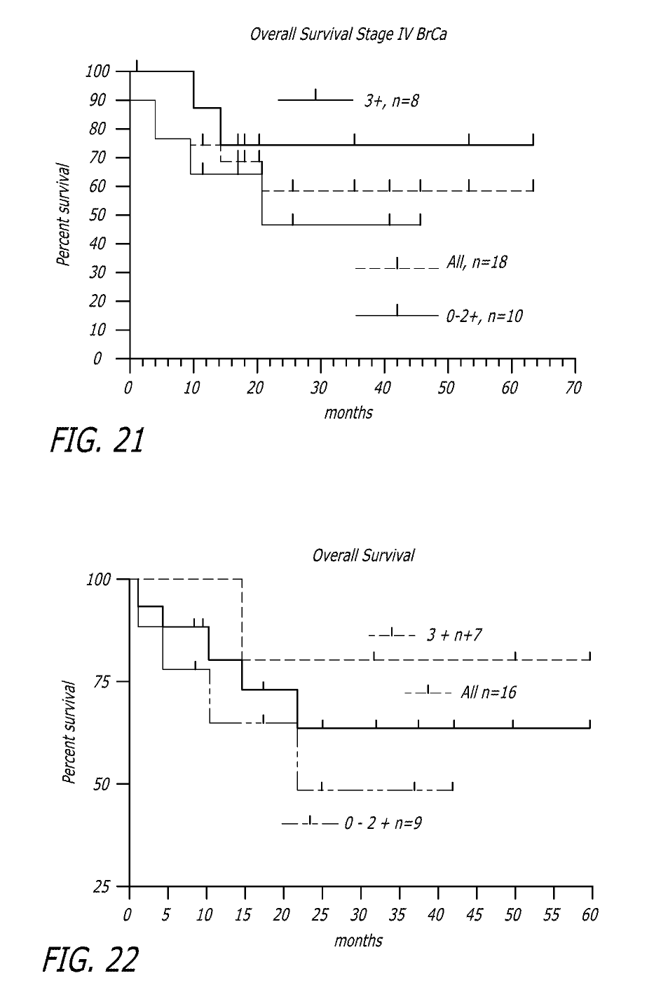

As shown in FIG. 18, IFNγ EliSpots were detected during immunotherapy. FIG. 19 shows persistent cytotoxicity directed at SK-BR-3 in fresh PBMC from patients. PBMC were acquired from whole blood collected at various time points over the course of treatment. All patient PBMC samples were tested for cytotoxic activity against HER2-expressing SK-BR-3 cells (25:1 E/T) and against HER2-negative Raji cells as a negative control. (Grabert et al., Clin Canc Res 12:569). FIG. 20 shows serum IL-12 levels in patients. FIG. 21 shows overall survival of Stage IV BrCa. ATC can be expanded ex vivo using anti-CD3 and IL-2 and ATC can be armed ATC.

The results of this Example are summarized as follows. Her2Bi armed ATC and low dose IL-2 and GM-CSF can be safely given up to a dose total dose of 160 billion in 8 divided doses over 4 weeks. Specific cytotoxicity directed at SK-BR-3 target can be detected in the peripheral blood of the patients up to 4 months after immunotherapy. Type I cytokine patterns (IFN, TNF, GM-CSF) and IL-12 are induced by the infusion of armed ATC. Overall survival data suggests that infusions of targeted T cells in immunocompetent patients vaccinate the patient against their own breast cancers.

### Example 6

### Targeting Protocol to Induce Anti-Tumor Immunity

Applicants' previous studies show that anti-CD3 activated T cells (ATC) from peripheral blood could be expanded in IL-2 for 14 days and armed with anti CD3×anti-Her2/neu bispecific antibody (Her2Bi) to kill Her2/neu tumor targets on breast, prostate, pancreatic, and ovarian cancers. Applicants' preclinical studies show that Her2Bi armed ATC exhibit high levels of specific cytotoxicity, kill multiple times, secrete cytokines and chemokines upon repeated re-exposures to Her2/neu positive target cells. In this study applicants combined a targeting protocol to induce anti-tumor immunity that can then be transferred via re-expanded ATC into patients after stem cell transplant.

Clinical trial results for Her2Bi targeted ATC are summarized as follows. (1) ATC can be expanded ex vivo using anti-CD3 and IL-2 and ATC can be armed ATC. (2) Her2Bi armed ATC and low dose IL-2 and GM-CSF can be safely given up to a total dose of 160 billion in 8 divided doses over 4 weeks. (3) Specific cytotoxicity directed at SK-BR-3 target can be detected in the peripheral blood of the patients up to 4 months after immunotherapy. (4) Type I cytokine patterns (IFN, TNF, GM-CSF) and IL-12 are induced by the infusion of armed ATC. (5) Overall survival data suggests that infusions of targeted T cells in immunocompetent patients vaccinate the patient against their own breast cancers.

The in vitro findings for ATC armed with Her2Bi are summarized as follows. (1) Arming with HER2Bi creates artificial antibody receptor ATC that targets Her2/neu tumor targets. (2) They kill up to 5 times in vitro. (3) The secrete IL-2, IFNγ, TNFα, and GM-CSF and chemokines upon binding to targets. (4) They proliferate after killing tumor targets. (5) They prevent the development of xenografted tumors in Beige SCID mice and induce remissions when used to treat mice with established prostate tumors. (6)>95% CD3+ cells, 60-80% CD8 cells, and 20-40% CD4 cells.

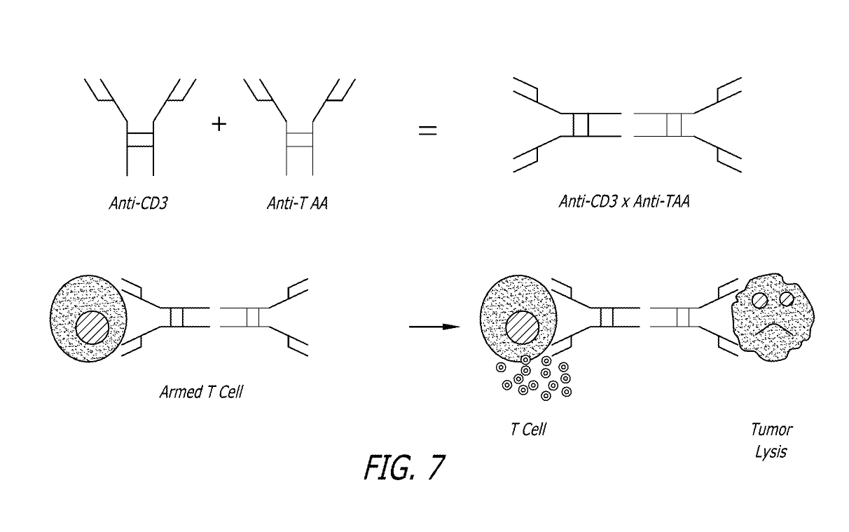

FIG. 7 shows the concept in which two mAbs are heteroconjugated and then used to arm ex vivo expanded ATC. The ATC armed with anti-CD3×anti-TAA (Her2=TAA) bind with the TAA on the surface of tumors leading to cytotoxicity and cytokine/chemokine release.

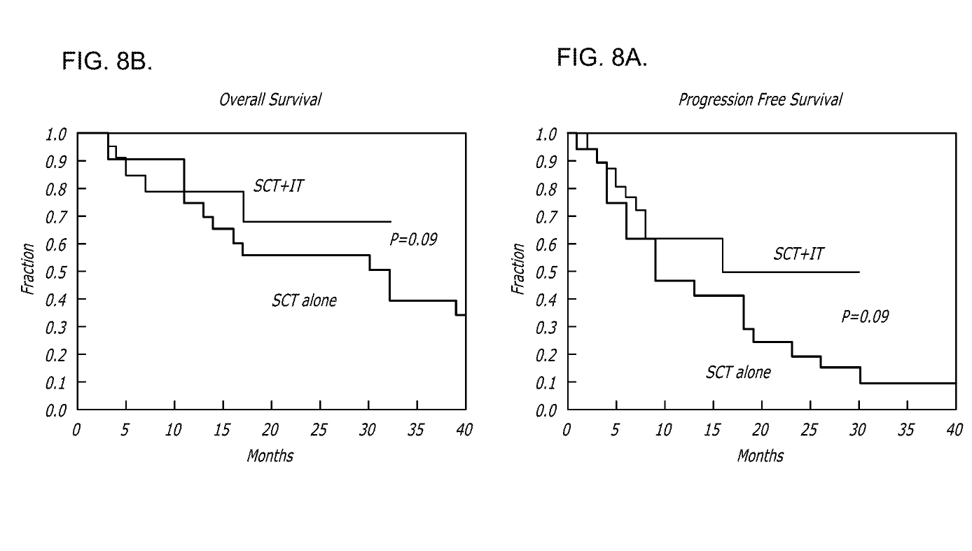

As shown in FIGS. 8A and 8B, multiple infusions of ATC in combination with low dose IL-2, and GM-CSF given as immune consolidation after SCT for Stage VI BrCa that may improve overall survival, as shown in FIG. 8A, and progression free survival, as shown in FIG. 8B. Engraftment occurred promptly and there were no infections or regimen related toxicities. All deaths were due to relapse.

The aims of the present Example were to determine whether the combination of: (1) infusions of Her2Bi armed anti-CD3 activated T cells (ATC) from women with stage IV metastatic breast cancer and (2) multiple infusions of unarmed ATC) expanded from leukopheresis after infusions of Her2Bi armed ATC, can transfer and boost anti-breast cancer immunity when given after stem cell transplant for stage IV breast cancer. Immune function tests are performed to evaluate immune responses after stem cell transplant. Production of armed or unarmed ATC is carried out as follows: PBMC are obtained from pheresis and treated with OKT3 (20 ng/ml)+100 IU/ml of IL-2; ATC are split every other day, then harvested, armed with BiAb or left unarmed, and cryopreserved. Quality control is conducted for seven days.

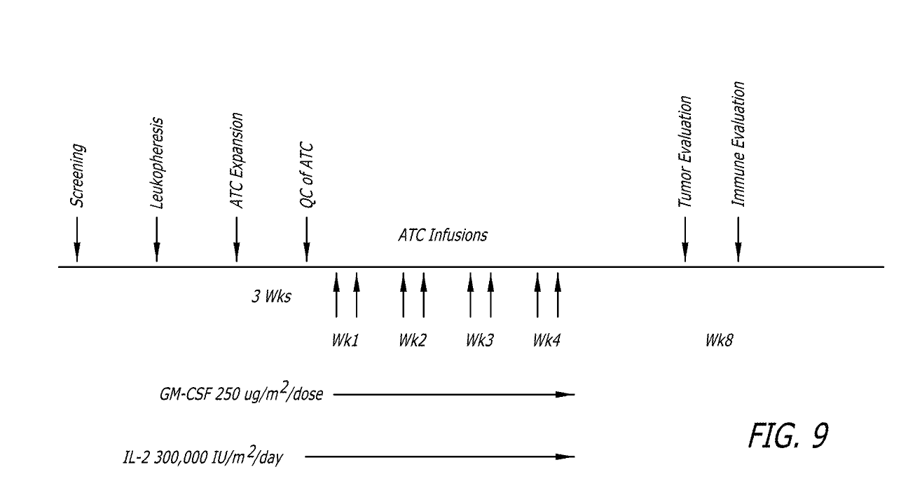

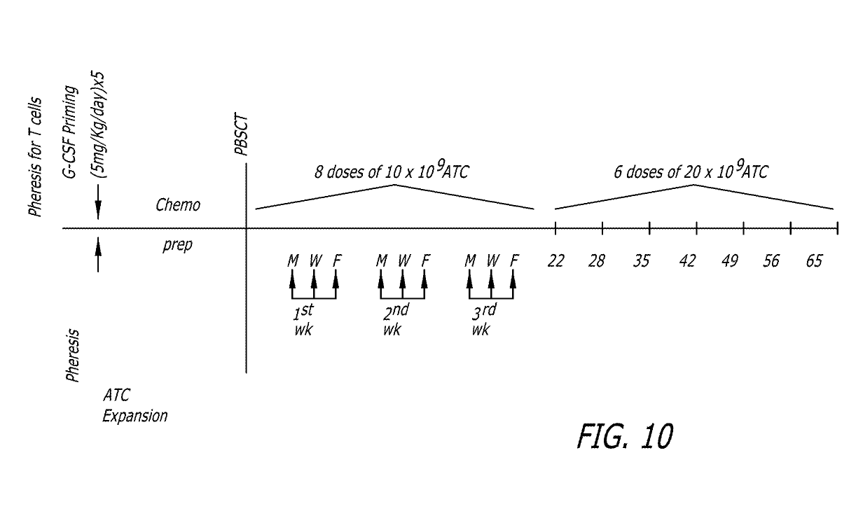

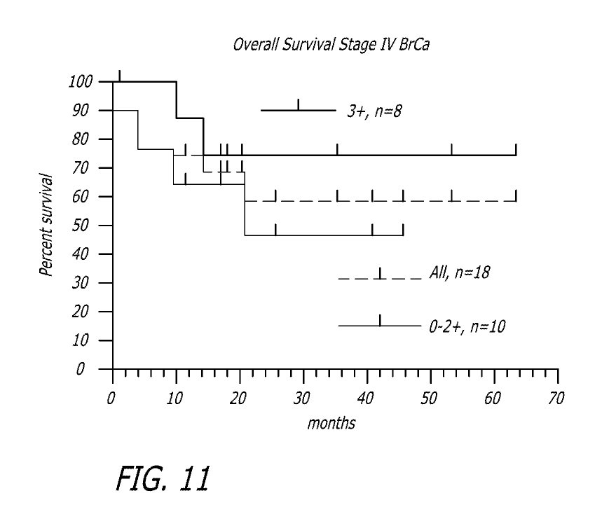

HerBi armed ATC vaccination protocol (first part of protocol) is shown in FIG. 9. ATC infusions after PBSCT (second part of protocol) are shown in FIG. 10. The results of this Example suggest that targeting with Her2Bi armed ATC to induce anti-tumor immunity can be transferred via re-expanded ATC into patients after stem cell transplant. In both patients, applicants were able to increase specific cytotoxicity directed at SK-BR-3 when ATC were expanded after the vaccination step. In 1 of 2 patients, applicants were able to detect cytotoxicity directed at SK-BR-3 after 9 infusions of ATC (˜3 weeks after SCT). These data suggest that boosting anti-tumor immunity prior to SCT and expanding the anti-tumor immunity and re-infusing T cells after SCT may provide an anti-tumor effect. As shown in FIG. 11, overall survival data in 18 patients suggests that infusions of targeted T cells in immunocompetent may improve overall survival. There were eight patients who were Her3+ and ten patients with Her2 0-2+. All of the patients had multiple rounds of chemotherapy and had therapy after immunotherapy. Most of Her2 3+ patients had Herceptin prior to immunotherapy.

### Example 7

### Expansion and Targeting of Anti-CD3 Activated Cord Blood T Cells

Applicants' previous studies show that anti-CD3 activated T cells (ATC) from peripheral blood could be expanded in IL-2 for 14 days and armed with anti CD3×anti-Her2/neu (Her2Bi), anti-CD3×anti-CD20 (CD20Bi), or anti-CD3×anti-EGFR bispecific antibody to kill Her2/neu, CD20, and EGFR+ tumor targets, respectively. This Example was performed to determine whether anti-CD3 activated cord blood T cells (CBATC) from banked or fresh cord blood could be expanded and targeted to tumors or hematologic malignancies for infusions after unrelated cord blood stem cell transplant (CBSCT). The specific aims of this Example were to determine if cord blood T cells can be expanded using anti-CD3 and IL-2; to determine the phenotype; and to determine if ATC armed with bispecific antibody (BiAb) can target and lyse Her2/neu or CD20 positive targets.

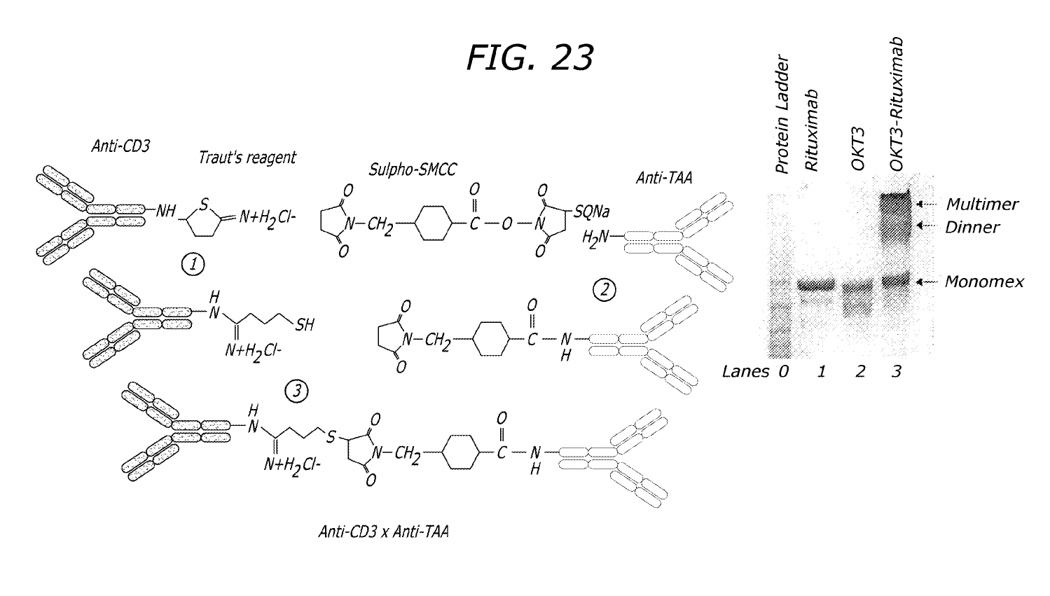

FIG. 23 shows heteroconjugation of anti-CD3 and anti-CD20. Rituximab (Rituxan®; Genentech) was heteroconjugated to anti-CD3 (OKT3) to produce the BiAb, CD20Bi. Briefly, OKT3 (1-5 mg) in 50 mM NaCl, 1 mM EDTA, pH 8.0 was reacted with a 5-10 fold M excess of Traut's reagent (2-iminothiolane HCl, Pierce). Rituximab (1-5 mg) in 0.1 M sodium phosphate, 0.15 M NaCl at pH 7.2 was reacted with a 4-fold molar excess of sulphosuccinimidyl 4-(N-maleimidomethyl)cyclohexane-1-carboxylate (Sulpho-SMCC) at room temperature for 1 hr. Both antibodies were then purified on PD-10 columns (Pharmacia, Uppsala, Sweden) in PBS to remove unbound cross-linker. The cross-linked mAbs were mixed immediately at equimolar ratios and conjugated at 4° C. overnight. The products of the heteroconjugation were resolved by SDS-PAGE (4-15% gradient) and detected by staining with Gelcode Blue by densitometric quantitation, CD20Bi resolved as 26% monomer, 32% dimer, and 42% multimer fractions.

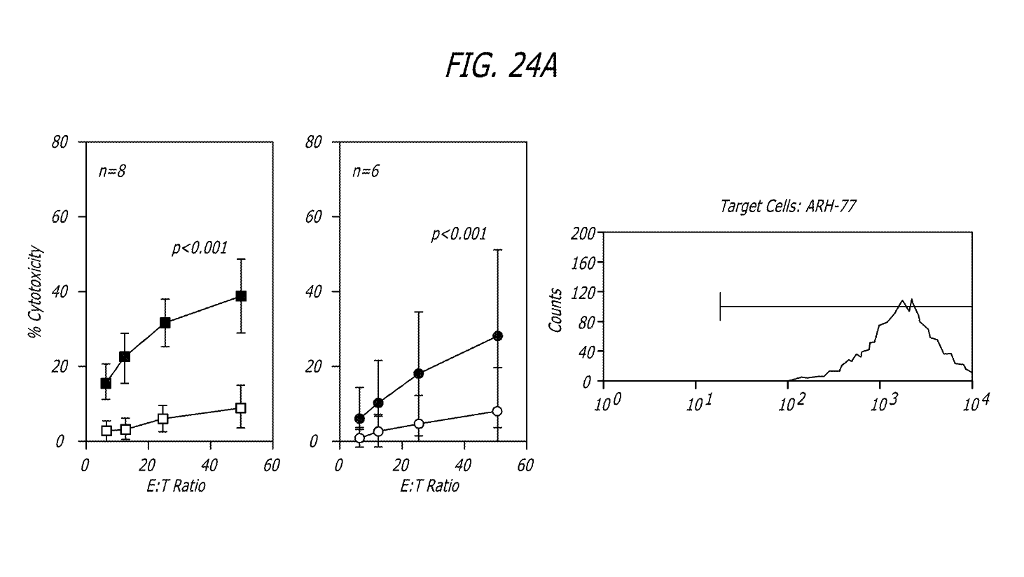

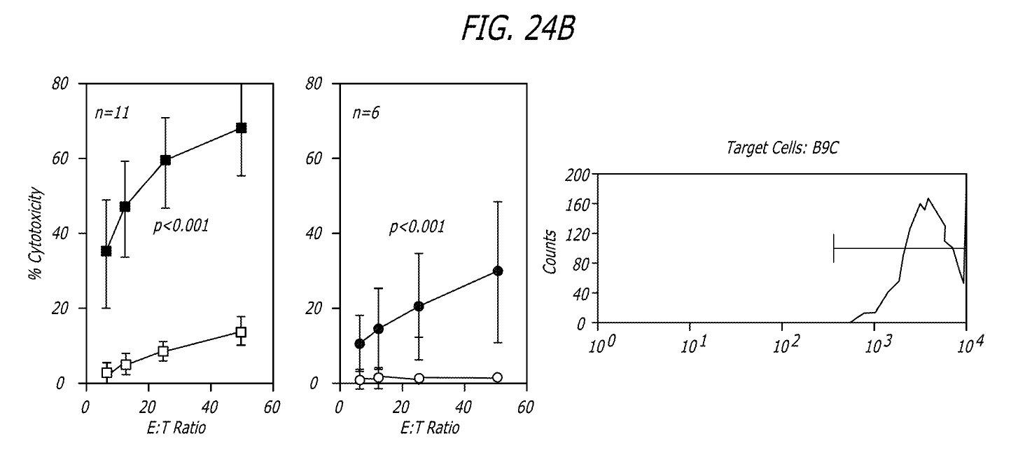

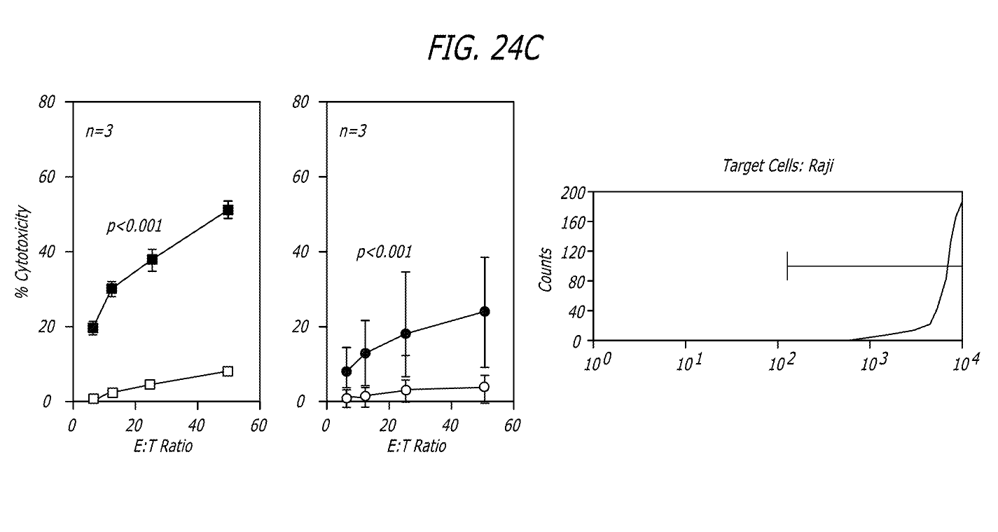

FIGS. 24A-C show cytotoxicity mediated by CD20Bi armed ATC from peripheral blood of normals and cancer patients in various target cell lines. FIG. 24A shows the results in an ARH-77 target cell line. FIG. 24B shows the results in a B9C target cell line. FIG. 24C shows the results in a Raji target cell line. Specific cytotoxicity profiles of CD20Bi-armed activated T cells (ATC) from normal donors and cancer patients compared to their unarmed ATC as a function of CD20 expression in target cell lines. ATC (□, ∘) were armed (, •) at 50 ng CD20 B per million ATC and evaluated by cytotoxicity assays. Results are expressed as the mean (±SD) cytotoxicity for the indicated population (n=as indicated) of normal donors (squares) or patients (circles). Flow cytometry panels (right) show binding of CD20Bi (black line) compared to binding of anti-CD20 (dark gray line) or IgG2a-PE isotype control antibody (light gray line) to the indicated cell line.

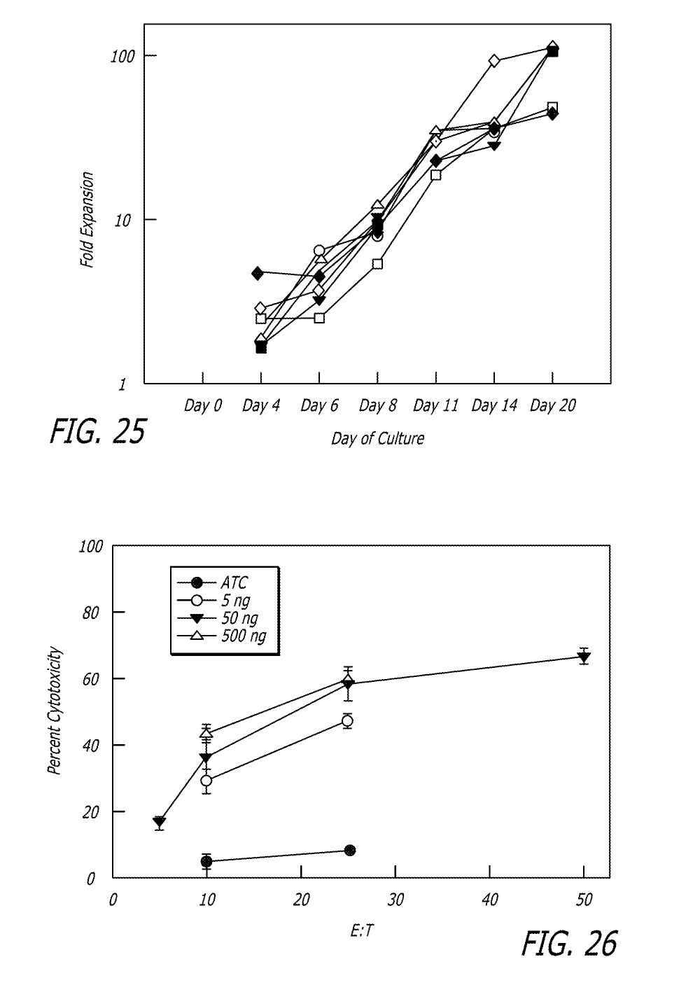

FIG. 25 shows anti-CD3 stimulated expansion of CB ATC. Fold expansion of anti-CD3 activated CB: CB mononuclear cells were activated with 20 ng/ml of OKT3 and expanded in IL-2 Eight frozen CBMC were thawed, activated, washed, and grown in IL-2 up to 20 days. FIG. 26 shows arming doses of anti-CD3×anti-Her2 BiAb. Cytotoxicity mediated by cord blood ATC armed with increasing doses of OKT3×Herceptin (Her2Bi) was performed as follows. Frozen CBMC were thawed and grown in IL-2 of 13 days. ATC were armed with 0, 5, 50 and 500 ng/106 cells of Her2Bi as indicated. 51Cr release assay was performed to determine cytotoxicity against SK-BR-3 target cells at different arming doses and E:T.

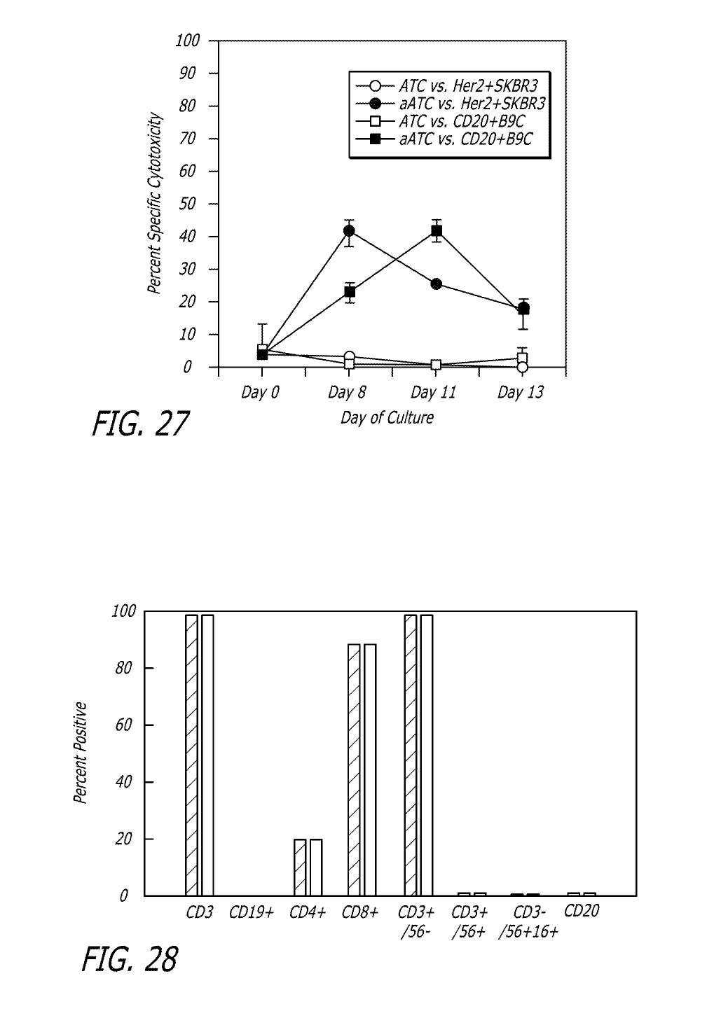

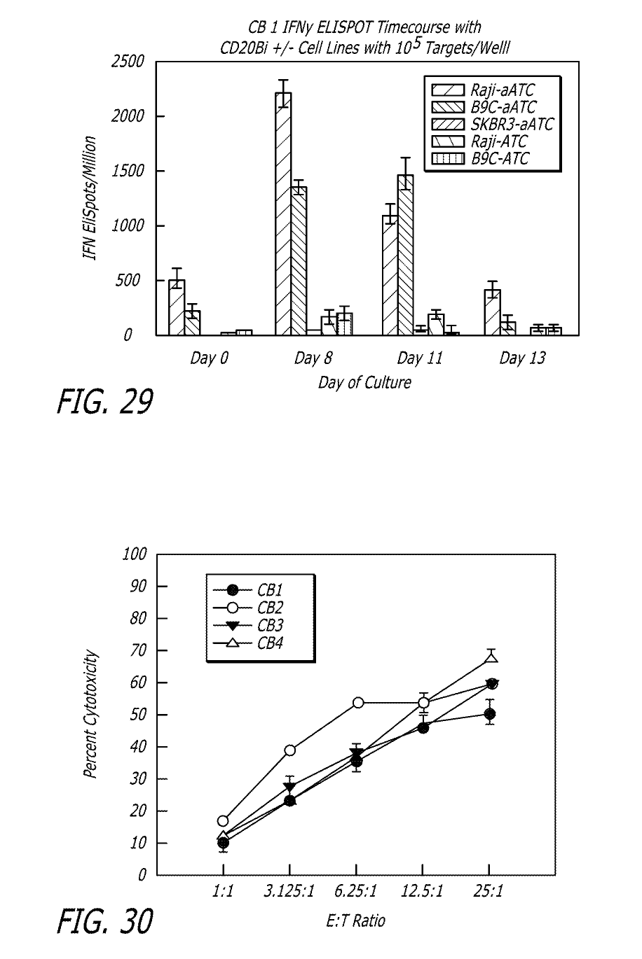

FIG. 27 shows the time course of armed ATC cytotoxicity. Time course of the development of Her2B1 and CD20Bi cytotoxicity directed at SK-BR-3, B9C, and Daudi targets, respectively, was performed as follows. CBATC were harvested at the indicated days and armed with 50 ng/million of Her2B1 or CD20Bi and tested in 51Cr release specific cytotoxicity against SK-BR-3 and Raji cells. FIG. 28 shows phenotyping on 2 CB after thirteen days. FIG. 29 shows IFN EliSpots induced by Her2 or CD20 binding. CD20Bi Armed ATC or unarmed ATC were tested in a time course to determine the optimal time for IFN EliSpots when targeting CD20+ cells and Her2/neu+SK-BR3 cells. EliSpots were developed after exposing CBATC or CD20Bi-armed CBATC to Raji, B9C or SK-BR3 targets. Unstimulated CBATC were <50 cells/million (not shown). Data are expressed as IFN EliSpots per million cells plated.

FIG. 30 shows specific cytotoxicity mediated by Her2Bi armed ATC as a function of various E:T ratios. Cytotoxicity Mediated by 4 Her2Bi-CB Armed ATC at Various E:T. was determined as follows. ATC were produced from cryopreserved CB. CB ATC were armed with 50 ng/million and cytotoxicity was done against SK-BR-3 targets. Unarmed ATC (data not shown) was consistently less than 4.5%.

In summary, CB ATC can be expanded ex vivo using anti-CD3 and IL-2. CB ATC can be armed with Her2B1 and CD20Bi to mediate high levels of specific cytotoxicity directed at Her2 and CD20 positive targets. CBATC secrete IFN when specifically targeting Her2/neu or CD20 positive targets. Armed CBATC can be used in immunotherapy approaches to target both solid tumors and CD20+ hematologic malignancies after stem cell transplant. This strategy may be useful after stem cell transplant when used in combination with a second cord in double cord blood transplants.

### Example 8

### HER2BI-Armed Targeted T Cell Studies

In preclinical studies, applicants have shown that Her2Bi, created by heteroconjugating anti-HER2 (Herceptin®; purchased from Genentech) to anti-CD3 (OKT3, Orthobiotech) can be used to arm ATC that have been cultured for 8-16 days. Specific cytotoxicity of ATC armed with Her2Bi (50 ng/10⁶ ATC; dose-titration optimized) was shown against HER2-expressing cell lines derived from breast cancers (SK-BR-3, MCF-7)¹, pancreatic cancers (MIA PaCa-2, COLO 356/FG)⁴⁸, and prostate cancers (LNCaP, DU 145, PC-3).⁴⁹ At effector:target ratios (E/T) from 3:1 to 50:1, both Her2Bi-armed normal and patient ATC were significantly more cytotoxic against HER2-expressing cells than ATC, anti-HER2, anti-CD3 alone, or ATC armed with irrelevant BiAb directed at CD20+ targets. Her2Bi-armed ATC also secreted significantly higher levels of some T_{H1}/T_{H2} cytokines compared to ATC alone. In mice, intravenous infusions of Her2Bi-armed ATC significantly delayed growth of established PC-3 tumors compared to mice that received ATC alone or vehicle (p<0.001) without inducing toxicities⁴⁹. In studies evaluating the long-term activity and fate of Her2Bi-armed ATC, expansion and division of Her2Bi-armed ATC vs. unarmed ATC have been determined by comparing survival and ability of cells to divide and kill target cells when repeatedly exposed in vitro to SK-BR-3 cells over 336 h of culture. Up to 2 weeks after a single arming, ex vivo expanded Her2Bi-armed ATC co-cultured with SK-BR-3 targets increase in number, undergo multiple cell divisions, mediate specific cytotoxicity, and secrete both cytokines and chemokines without undergoing activation-induced cell death⁵⁰.

Importantly, Her2Bi armed ATC mediate high levels of cytotoxicity against MCF-7 cells, a cell line expressing very low levels of HER2 receptors/cell (<1.0×10⁴), considered negative by IHC. Redirected T cell targeting to low level receptors is most likely responsible for enhanced MCF-7 killing since Herceptin® mediated cell death due to interference with receptor-mediated pathways is ineffective. Therefore, this therapy would potentially be effective in clinical situations (˜80% of patients) wherein patients have low level HER2 (0-2+) MBC expression (a characteristic that precludes Herceptin® therapy).

### Example 9

### Preliminary Results from the Phase I Clinical Trial Using HER2Bi-Armed ATC to Target Metastatic Breast Cancer (MBC)

A phase I dose escalation clinical trial is being done in women with stable to progressive HER2-positive or HER2-negative MBC to determine the maximum tolerated dose (MTD) for Her2Bi-armed ATC given with IL-2 and GM-CSF. Seventeen women (41% Her2 3+ and 53% Her2 0-2+) with a median age of 50 years (31-68 years) were enrolled from September 2 to February 7. T cells for this trial, derived from a pheresis product, were activated with anti-CD3 and cultured in RPMI supplemented with 2% human serum and IL-2 for 14 days. ATC were harvested, armed with Her2Bi, cryopreserved, and infused in 8 divided doses. Testing of the cell product showed that mean cytotoxicity (±SD) induced by patient's Her2Bi-armed ATC (59.3%±19.2) was significantly greater (p<0.0001) than that induced by patient's unarmed ATC (3.0±2.9) before immunotherapy (IT). The mean % CD3+, CD4+, and CD8+ cells that were grown, harvested, and armed were 86.7±13.5, 52.4±15.2, and 34.6±15.0, respectively. There was an inverse correlation between the in vitro cytotoxicity of patient's Her2Bi-ATC and the proportion of CD4+ cells in the product [Spearman=−0.55, p=0.03 for CD4]. This finding is consistent with applicants' in vitro data¹

The MTD of Her2Bi-armed ATC has not been reached. The MTD is defined as the dose below the dose at which dose limiting toxicity occurred in 2 of 6 patients. The highest dose level completed is 20×10⁹ Her2Bi-armed ATC per infusion (160×10⁹ total dose of armed ATC). The most frequent side effect experienced by patients was Grade 3 chills. Grade 3 headaches emerged as the second most common side effect. Fewer than 50% of the patients enrolled experienced each of the remaining symptoms. By episode per infusion, incidence of chills and headache at dose level 1 (8.6% and 3.1%, respectively) increased for dose level 2 (20.8% and 8.3%, respectively) and then again at dose level 3 (43.1% and 19.6%, respectively). One patient at dose level 3 experienced headache and hypertension associated with a subdural hematoma (grade 4) thought to be related to the infusions. Consequently, an additional 3 patients were added to the dose level which, in turn, was successfully completed without further dose limiting toxicities. One patient has been accrued at the last and highest dose level of 40×10⁹ Her2Bi-armed ATC per infusion (320×10⁹ total dose).

Applicants have determined the clinical responses to Her2Bi-armed ATC. There was one PR in a woman with a liver metastasis and more than half of the patients remain stable with no evidence of increase in tumor size or development of new lesions at their follow-up, one month after completion of the last Her2Bi-armed ATC infusion. Serum tumor markers exhibited impressive decreases in Carcinoembryonic Antigen (2 of 4 >50% decrease and 2 with 15-50% decrease), CA 27.29 (3 of 3 had >15-50% decrease), and Her2 (2 of 5 with >50% decrease and 2 of 5 with a 15-50% decrease) in the serum within a month after the last infusion.

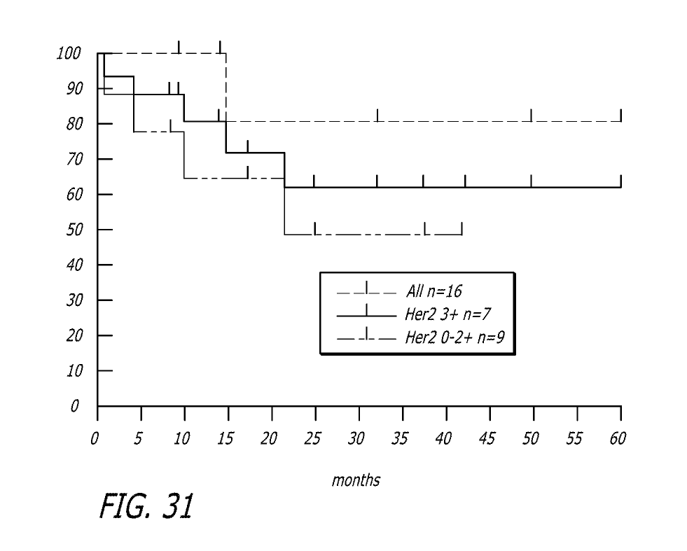

The overall survival of patients treated with Her2Bi-armed ATC is presented in FIG. 31. Two patients were excluded from analyses; one patient who died before 1 month and the other with an unknown HER2 status. In the HER2(3+) group, median overall survival has not been reached. The median survival for the HER2(0-2+) cohort is 21.3 mos. The median survival for all of the patients remains undefined with >70% of patients surviving.

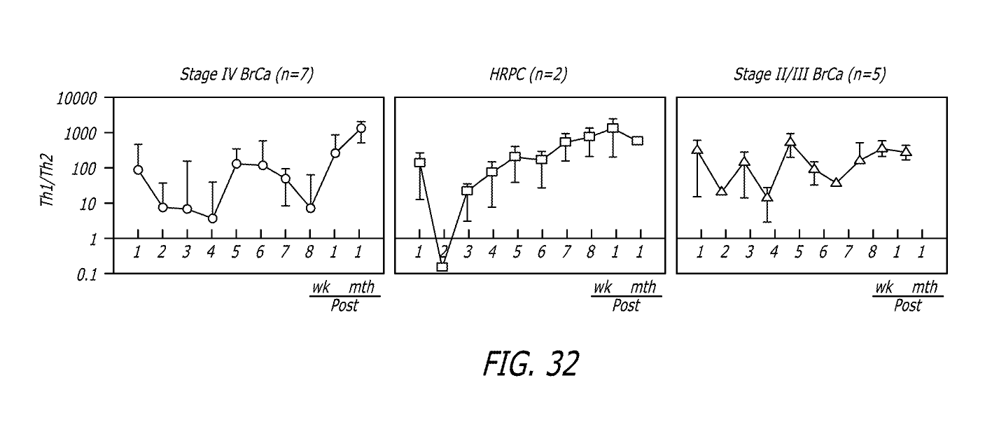

Applicants analyzed the serum cytokine responses in patients given Her2Bi-armed ATC. Serum samples, taken at indicated time points over the course of armed ATC infusions, were tested for cytokines (IL-2, IL-4, IL-5, IL-10, IL-12 p70, IL-13, GM-CSF, IFNγ, and TNF-α) representative of Th1- and Th2-type immune responses using the Bio-Plex Protein Array System (FIG. 32). The three panels of FIG. 32 show data from MBC [left], Hormone Refractory Prostate Cancer (HRPC)[middle], and Stage II/III BrCa [right] patients as a function of infusion number (inf #). These results, summarized as a calculation of the mean Th1[IL-2+IFNγ]/Th2[IL-4+IL-5] ratio, show a Th1-type response induced as function of armed ATC infusions, increasing from 89.1 at Inf #1 to 538.6 at Inf #8. These findings were consistent with increased specific IFNγ production observed by EliSpot analysis of patient post-Inf PBMC exposed to HER2-positive SK-BR-3 tumor cells. These observations show that the overall immune status of cancer patients receiving armed ATC shifts towards an anti-tumor Th 1 response as a function of armed ATC infusions.

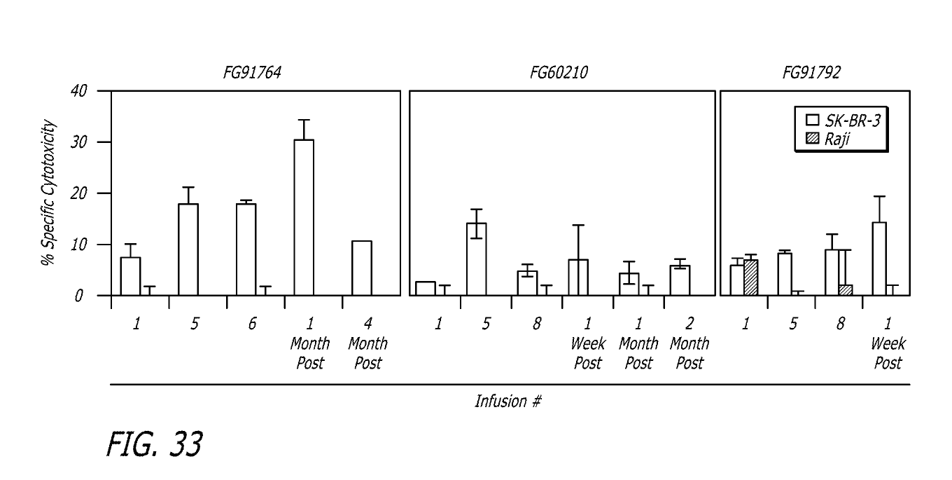

FIG. 33 shows specific cytotoxic activity observed in patient PBMC following treatments with Her2Bi-armed ATC. Five patients undergoing Her2Bi-armed ATC infusions were studied for cytotoxicity directed at SK-BR-3 before, during, and after treatment. FIG. 33 shows 3 representative patients. A significant increase in specific cytotoxicity directed at SK-BR-3 cells by PBMC obtained from all patients was observed during the course of treatment, with peak levels ranging from 14.2±3% to 30.7±0.5%. Although there were variations between time points, specific cytotoxicity tended to increase during treatment with 2 of 5 patients exhibiting peak cytotoxicity just before or following infusion 8. Remarkably, 1 week after completion of the 8th armed ATC infusion, PBMC from 3 of 5 patients maintained cytotoxicity ranging from 14.7±2.0% to 17.4±0.4%. Cytotoxicity mediated by PBMC obtained 1-week post infusion 8 from 2 of 3 of these patients was significantly (P<0.05) specific for SK-BR-3 cells compared with Raji cells (CD20+ controls). More importantly, cytotoxicity in patient's PBMC persisted up to 4 months after armed ATC infusions directed at HER2 over-expressing cells. These data suggest that armed ATC may persist and function in vivo such that they might provide a cumulative anti-tumor effect.

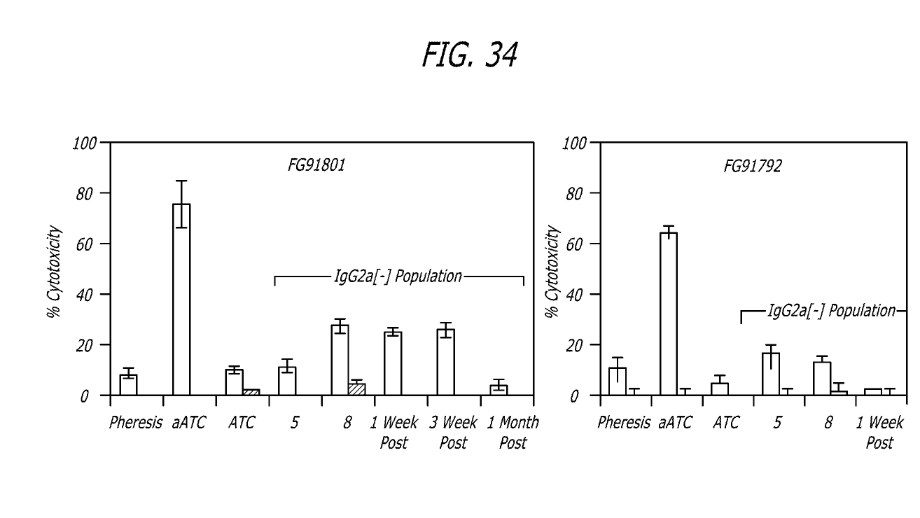

FIG. 34 shows the enhanced specific cytotoxicity that was mediated by endogenous lymphocytes from patients. PBMC were obtained from BrCa patients at the time of pheresis to obtain leukocytes for ATC expansion, prior to the 5th, 6th and 8th infusions, and then at the indicated time points after armed ATC infusions (FIG. 34). In some patients, infusions of Her2Bi-armed ATC correlate with induction of specific cytotoxicity. Moreover, it was observed that enriched IgG2a-(endogenous) cell populations exhibited significant cytotoxic activity.

FIG. 20 shows that patients receiving Her2Bi-armed ATC infusions had increased levels of IL-12. IL-12, produced mainly by activated monocyte/macrophages, is the principle cytokine for polarizing T cell responses towards a Th1 phenotype. Furthermore, IL-12 has been shown to enhance the cytotoxic functions of NK and CD8+ T cells. Applicants have observed an increase in serum levels of IL-12 occurring around 2 weeks after initiating armed ATC infusions in patients with MBC (FIG. 20) and hormone refractory prostate cancer. These data are critical in that they suggest 1) armed ATC infusions establish a systemic Th1-type anti-tumor milieu and 2) endogenous cells of the monocyte/macrophage lineage are activated to produce IL-12 under conditions elicited by armed ATC infusions. The results for IL-12 support the induction of endogenous immunity since IL-12 is not produced by T cells, therefore, it cannot be ascribed to the infused armed ATC.

To determine whether human anti-mouse antibody (HAMA) responses played a role in clearing armed ATC applicants examined the patients' sera. Because one mAb moiety is an unmodified mouse mAb, applicants evaluated patients' sera for the development of HAMA. Of 11 patients evaluated, none developed clinically significant HAMA levels (>10 ng/ml) during or immediately following the treatment regimen. Average HAMA concentrations in patients did not differ significantly as function of dose level (p=0.55). There were no correlations between levels of IgG2a+ cells in patients' peripheral blood and their respective HAMA levels (r²=0.001; p=0.86); therefore, HAMA responses are not likely to play a role in clearance of armed ATC.

In summary applicants' phase I dose-escalation study shows that Her2Bi-armed ATC infusions are feasible, safe, induce in vivo anti-tumor CTL activity in both Her2+ and negative patients, can traffic to disease sites, and induce decreases in serum tumor markers in HRPC and BrCa patients. One PR was observed with a strong suggestion of prolonging overall survival in heavily pretreated with stage IV BrCa patients. The infusions did not induce HAMA responses, but induced a well-defined shift in the Th1/Th2 cytokine patterns in the serums of MBC, HRPC, and Stage II/III BrCa patients was associated with a 3-4 log increase in serum IL-12 after the 4th infusion.

### Example 10

### The Following Cell Lines were Used for Preliminary Studies

The cell lines used in these studies were: CD20+ cell lines—B9C (an immortalized normal B cell line), Raji (Burkitt's lymphoma), ARH-77 (multiple myeloma, Rituxan® resistant by CMC pathway) and the CD20− cell line K562 (chronic myelogenous leukemia).

### Example 11

### CD20BI-Armed Targeted T Cells Studies

FIG. 23 shows the production of heteroconjugated CD20Bi (OKT3×Rituxan®) via resolution by SDS-PAGE and staining with Gelcode Blue. Rituximab (Rituxan®; Genentech) was heteroconjugated to anti-CD3 (OKT3) to produce the BiAb, CD20Bi. Briefly, OKT3 (1-5 mg) in 50 mM NaCl, 1 mM EDTA, pH 8.0 was reacted with a 5-10 fold M excess of Traut's reagent (2-iminothiolane HCl, Pierce). Rituximab (1-5 mg) in 0.1 M sodium phosphate, 0.15 M NaCl at pH 7.2 was reacted with a 4-fold molar excess of sulphosuccinimidyl 4-(N-male-imido-methyl)cyclohexane-1-carboxylate (Sulpho-SMCC) at room temperature for 1 hr. Both antibodies were then purified on PD-10 columns (Pharmacia, Uppsala, Sweden) in PBS to remove unbound cross-linker. The cross-linked mAbs were mixed immediately at equimolar ratios and conjugated at 4° C. overnight. The products of the heteroconjugation were resolved by SDS-PAGE (4-15% gradient) and detected by staining with Gelcode Blue (FIG. 23, panel b) by densitometric quantitation, CD20Bi resolved as 26% monomer, 32% dimer, and 42% multimer fractions.

Applicants determined that arming ATC with CD20Bi redirects non-MHC restricted cytotoxicity via CD20 antigen binding and enhances lysis of CD20+ cell lines. The degree of binding of CD20Bi to ATC was evaluated using phycoerythrin-conjugated (PE) goat anti-mouse IgG2a to detect OKT3 and IgG. Dual staining was performed using anti-IgG1 and anti-IgG2a specific reagents to demonstrate specific binding of the BiAb. CD20Bi binding correlated with high CD20 expression on B9C, Raji, and ARH-77 cells and low CD20 expression on K562 cells. Additionally, binding was uncompromised by the BiAb heteroconjugation procedure since binding of the BiAb was comparable to binding of free, unconjugated Rituxan® (data not shown). For CD20Bi dose titration studies (FIG. 1), normal donor ATC were armed at 100, 50, 25, 5, and 1 ng per 10⁶ cells, washed to remove excess Ab, added to ⁵¹Cr-labeled B9C targets (4×10⁴ cells/round-bottomed microwell) at the indicated E/T, and incubated for 4 hrs at 37° C. Specific lysis was calculated by measuring ⁵¹Cr release in cell supernatants using the formula: [% specific lysis=(test-spontaneous release)/(maximum release-spontaneous release)]. ATC armed with Her2Bi (50 ng/10⁶ cells) was used as an irrelevant control and unarmed ATC as a negative control. Results are expressed as the % specific lysis (mean±SD) from triplicate wells. Arming doses of 25, 50, and 100 ng CD20Bi/10⁶ ATC resulted in significantly improved cytotoxicity compared to ATC armed with 1 ng CD20Bi/106 ATC or Her2Bi (p<0.05) or unarmed ATC (p<0.01) and demonstrated an approximate ED50 (effective dose resulting in 50% cytotoxicity of target cells) at the 25:1 E:T. For subsequent evaluations, the applicants selected an arming dose of 50 ng CD20Bi/10⁶ ATC.

### Example 12

### Arming ATC with CD20Bi Enhances Lysis of CD20 Positive Hematologic Cell Lines

ATC from normal donors were armed with 50 ng/million of CD20Bi and tested for cytotoxicity against CD20 positive and negative targets. CD20Bi-armed ATC were incubated with target cells at an E:T of 6.25, 12.5, 25, and 50 to 1. ATC armed with anti-CD3×anti-Her2/neu BiAb (Her2Bi; 50 ng/10⁶ ATC) or unarmed ATC and ATC admixed with unconjugated rituximab (125 ng/10⁶ATC) were used as an irrelevant control or negative control, respectively. Normal donor and patient-derived ATC armed with CD20Bi (50 ng/10⁶ ATC) were evaluated for mediating specific lysis of B9C, Raji, ARH-77 (FIG. 24), and K562 cells (data not shown) in ⁵¹Cr release assays. Compared to unarmed ATC, CD20Bi-armed ATC from normal donors mediated significant (p<0.001) specific lysis against all three cell lines that was representative of CD20Bi binding (FIG. 24) in ARH-77, B9C, and Raji cells (CD20+ hematologic cell lines), but not K562 cells (CD20-hematologic cell line). In addition, cytotoxicity directed at the CD20⁻ non-hematologic SK-BR-3 cell line (background binding; data not shown) was not significantly enhanced over ATC-mediated cytotoxicity.

### Example 13

Cytotoxicity Mediated by CD20Bi-Armed ATC is not Blocked by Free Rituxan®

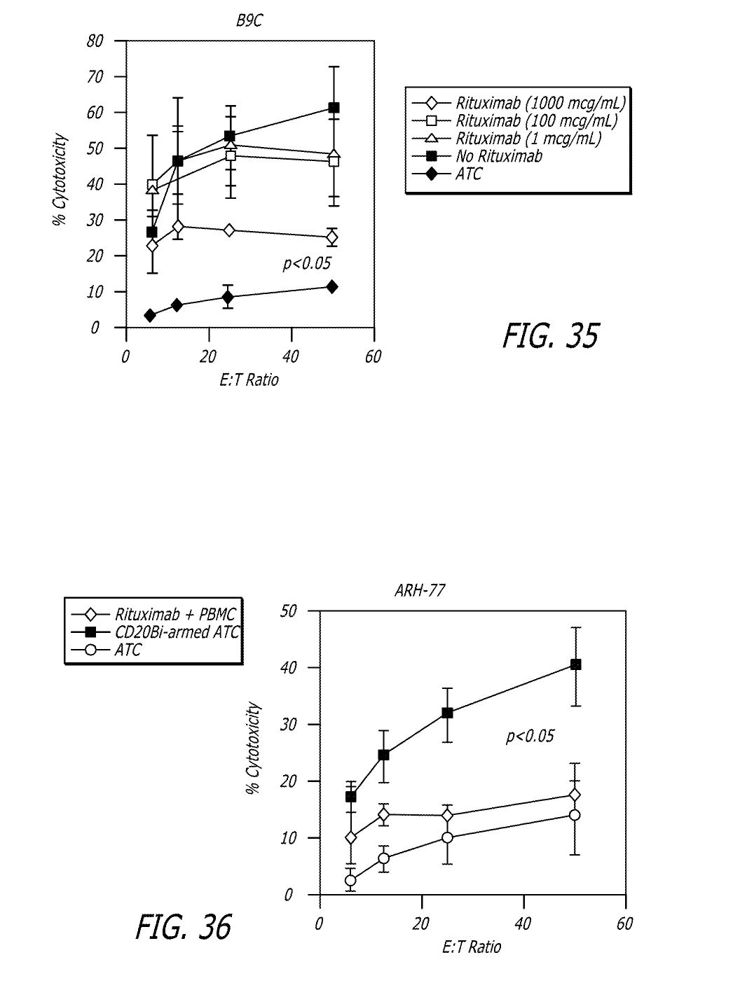

Cytotoxicity assays were performed, as described above, in the absence or presence of various concentrations (1-1000 μg/ml) of free, unconjugated Rituxan®. Normal donor ATC were armed CD20Bi at 50 ng per million cells. Unarmed ATC were the negative control. No blocking effect was observed until a Rituxan® dose of 1000 mcg/ml was reached. Results (FIG. 35), expressed as the % specific lysis (mean±SD) from triplicate wells, suggest that CD20Bi-armed ATC would be effective even in patients who may have been treated with and still have unconjugated Rituxan® in their circulation.

### Example 14

### ATC Armed with CD20Bi Mediate Cytotoxicity in Rituxan®-Resistant CD20+ ARH-77 Cells Comparable to Cytotoxicity in Other CD20+ Cell Lines, but not in the CD20− Cell Line, K562

Normal donor ATC were armed with CD20Bi (50 ng/10⁶ cells) and % specific lysis of B9C, Raji, ARH-77, and K562 cell lines was determined in ⁵¹Cr release assays as described above (FIG. 24). Compared to unarmed ATC, CD20Bi-armed ATC mediated specific lysis of all three CD20+ cells lines, even the ARH-77 cell line which is resistant to Rituxan® killing via the complement-dependent pathway⁵¹. In fact, when comparing complement-mediated (human plasma source) Rituxan® killing to CD20Bi-armed ATC-mediated killing (adjusting for equivalent concentrations of Rituxan® for each [2.5 μg/million cells]), CD20Bi-armed ATC induced significantly higher (p<0.0001) cytotoxicity against all targets tested and bypassed Rituxan® resistance in ARH-77 (FIG. 36). These results provide evidence that CD20Bi-armed ATC circumvent mechanisms of Rituxan® resistance to induce cytotoxicity in CD20+ cell lines.

### Example 15

### CD20Bi-Armed ATC Secrete Cytokines Upon Binding to Tumor Antigen

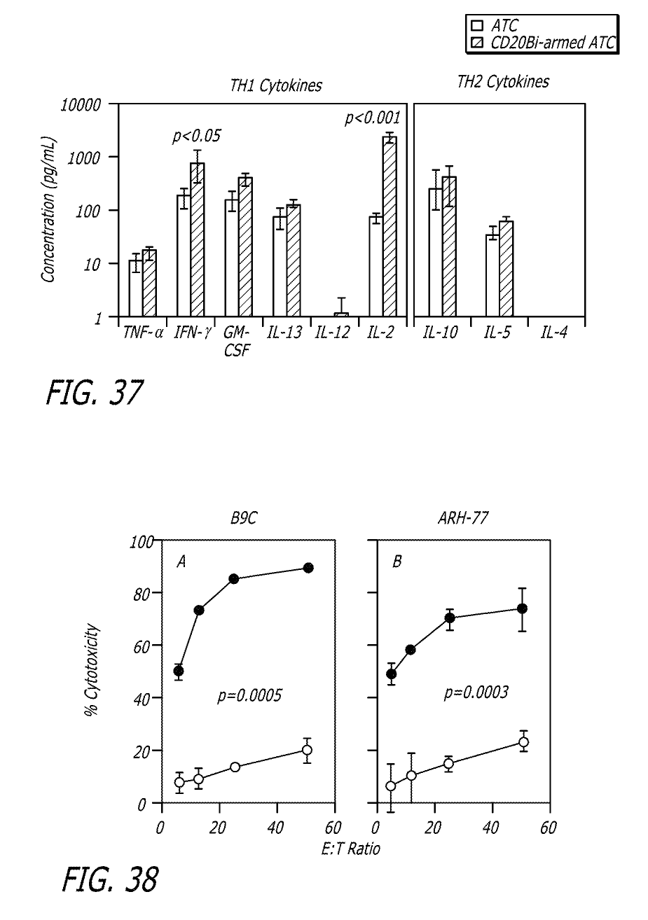

Since secretion of IFNγ and TNFα may provide anti-tumor effects, the applicants tested whether binding of CD20Bi-armed ATC to B9C targets at an E:T ratio of 10:1 would induce IFNγ and TNFα secretion. Normal donor ATC were armed with CD20Bi (50 ng/10⁶ cells) and plated onto B9C targets. Unarmed ATC from the same normal donor were plated in the same manner. The cells were incubated overnight at 37° C. and supernatants were tested for cytokines by ELISA. Results (FIG. 37), expressed as the cytokine concentration (mean±SD) from triplicate wells, suggest that CD20Bi-armed ATC secrete chemokines that could enhance trafficking of naïve T cells and APC to tumor lysis sites and be loaded with tumor antigens.

### Example 16

### NHL Patient's ATC Armed with CD20Bi are Highly Cytotoxic

Armed ATC from an NHL patient showed significantly enhanced cytotoxicity directed at B9C and ARH-77 compared to unarmed ATC (FIG. 38). The patient was previously treated with rituximab and cyclophosphamide, doxorubicin, vincristine, and prednisone (CHOP) followed by radiation to gastric lymphoma. These results compare favorable with findings in the MBC patients whose Her2Bi-armed ATC exhibit high levels of specific cytotoxicity.

### Example 17

### Preclinical Studies Targeting HER2/Neu and CD20 Using Cord Blood Cells

The applicants' preclinical and clinical data demonstrate the Her2B1 or CD20Bi-armed ATC exhibit high levels of specific cytotoxicity directed the Her2 and CD20 positive targets. In order to determine if armed CBATC could be developed and used clinically to augment GVL, applicants determine if CB cells could be armed and exhibit cytotoxicity in vitro.

In FIGS. 25 and 26, applicants showed that CB can be activated and expanded in vitro: In view of applicants' ability to expand ATC from peripheral blood for arming with Her2B1 and CD20Bi, Applicants asked whether ATC derived from cryopreserved cord blood units. As shown in FIG. 26, CB mononuclear cells could be activated with 20 ng/ml of OKT3 and consistently expanded in IL-2 for up to 20 days in culture (FIG. 25). These results show that cryopreserved umbilical cord can be expanded under the same conditions used to expand ATC from peripheral blood and CBATC can be consistently obtained from cryopreserved CB and can be used for immunotherapy.

### Example 18

### Dose Titration for Armed ATC with HER2Bi

Applicants confirmed that 50 ng/million of Her2Bi was optimal for mediating specific cytotoxicity. Since 50 ng/million was optimal for armed peripheral blood ATC, a dose titration was performed on CBATC using Her2Bi. FIG. 26 shows CB ATC that were left unarmed (ATC) and armed with 5, 50, and 500 ng/million ATC. CB ATC armed as well as peripheral blood ATC.

### Example 19

### Phenotyping of Two CB Before and after 13 Days of Culture

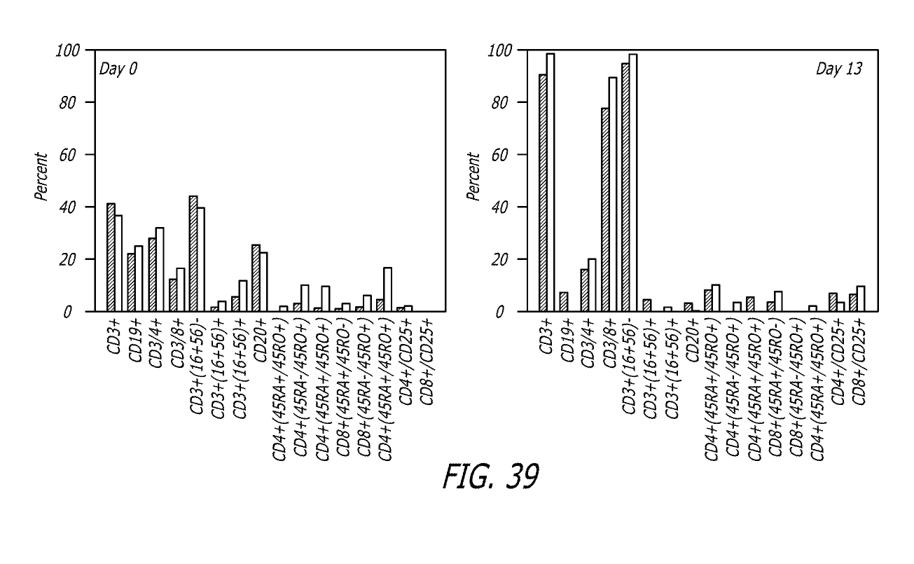

To determine the phenotype of CBATC, two cord blood units were activated with anti-CD3 and expanded in IL-2 for 13 days in culture. (FIG. 39) shows expansion of CD3+ cells and a slight increase in the proportion of CD8+ cells with a smaller proportion of CD4+ cells. CD25+ cells developed but remained less than 7% of the entire population. The numbers of CD56+ cells did not increase during the culture. A similar distribution of T cell subsets is observed following the expansion of peripheral blood.

### Example 20

### Development of HER2Bi and CD20Bi Armed Specific Cytotoxicity

Since applicants were able to demonstrate specific cytotoxicity of CBATC armed with Her2Bi against SK-BR-3, they tested the development of specific cytotoxicity for Her2/neu+ (SK-BR-3) and CD20+ (B9C) targets by testing PBMC and CBATC harvested on day 8, 11, and 13 that were armed with 50 ng/million of Her2Bi and CD20Bi, respectively. As shown in FIG. 27, optimal cytotoxicity of CBATC armed with Her2B1 or CD20Bi are observed after 8 and 11 days of culture, respectively. Cytotoxicity directed at SK-BR-3 and B9C (CD20+) targets was performed at E:T of 25:1. The time course is similar for armed CBATC and peripheral blood ATC.

### Example 21

### Cytotoxicity with CBATC Armed with HER2Bi is Highly Reproducible

Four CB units were thawed, activated and expanded for 14 days to produce CBATC, armed with Her2Bi and tested at the indicated E:T for cytotoxicity directed at SK-BR-3. All 4 randomly chosen CBATC samples were highly cytotoxicity to SK-BR-3 targets in the same range across all of the E:T tested, as shown in FIG. 30. In an extended culture experiment, mean cytotoxicity for Her2Bi-armed CBATC after 47 days was 36% at an E:T of 25:1 compared to 4.35% for unarmed CBATC. These results show that cryopreserved CB units can be consistently expanded ex vivo and armed with BiAb to induce tumor specific killing.

### Example 22

### Defining the Time Course and Specificity of Armed CBATC Cytotoxicity Using IFN EliSpots

By using EliSpots as a surrogate marker of cytotoxic T cells that secrete IFNγ, the number of cells secreting IFNγ can be tracked. CBATC armed with CD20Bi and unarmed ATC were co-cultured for 1 hr with the indicated target cells and the effector populations were plated at 100,000 cells per microwell. The time course shows specific for targeting for both CD20+ cells (B9C and Raji) and also SK-BR-3 cells. FIG. 29 shows that targeting CD20 armed CBATC results in a specific increase in IFNγ EliSpots for CD20+ targets (B9C and Raji). There were no responses during the culture period for unarmed ATC stimulated with Raji, B9C, and SK-BR-3. These data not only show specificity of the CD20Bi-armed CBATC but the responses peaked at day 8.

### Example 23

### ATC Derived from Adult Peripheral Blood Mononuclear Cells (PBMC) are Allo-Non-Responsive to Alloantigens

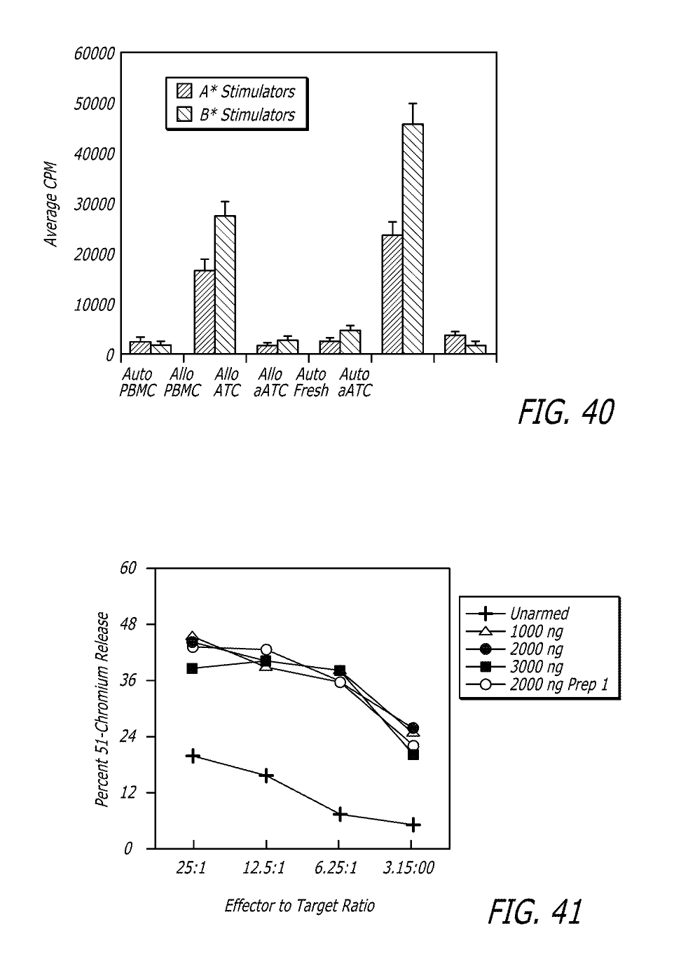

In order to determine whether ATC from peripheral blood would be relatively unresponsive to unrelated alloantigens, applicants stimulated PBMC, ATC, CD20Bi armed ATC, and fresh control T cells from two different normal donors with irradiated (2500 rads) unrelated PBMC. The results are shown in FIG. 40. Fifty thousand responders were placed into cultures with 50,000 irradiated PBMC. As expected auto PBMC is non-responsive to irradiated auto PBMC. The allo PBMC is the responsive MLC control wherein the responses are of individual B PBMC are responding to irradiated A PBMC and responses of individual A PBMC are responding to irradiated B PBMC. Allo ATC shows that ATCs from individual B or individual A are not responsive to irradiated PBMC from A or B, respectively. Allo Fresh T cells (purified T cell allo MLR control) shows that enriched T cells for individual B and A PBMC are very responsive to alloantigens. Auto aATC shows that autologous ATC armed with CD20Bi is non-responsive to alloantigens. In summary, these MLR data show that peripheral blood ATC or armed ATC are non-responsive to alloantigens in a MLC assay. This depressed responsiveness to alloantigens could translate into significantly less GVHD when peripheral blood ATC or armed ATC are given after alloSCT.

### Example 24

### CBMC and CBATC are Non-Responsive to Alloantigens in MLC Assays

In order to test the alloresponsiveness of CBMC and CBATC, CBATC derived from 4 frozen CB (1-4) were expanded and tested in MLC against irradiated allogeneic stimulator cells. All of the data have been normalized to 1.0=the allocontrol (A×B*) (FIG. 5). Allocontrol+CBATC was a co-cultured setup to determine if CBATC added to the allocontrol (A×B*) would suppress a third party mixed MLC. The mean % (±SD %) alloresponsiveness of CB to alloantigen was 32±7% of the allocontrol and the mean alloresponsiveness of CBATC to alloantigen was 17±5% of the allocontrol. The addition of CBATC back was 106±25% of the allocontrol.

### Example 25

### Anti-CD20 targeting of mouse CD19 lymphoma cells (A20) in BALB/c Mice

2C11×anti-CD19 (CD19Bi) was produced by heteroconjugation and used to arm 2C11 stimulated murine activated T cells derived from splenocytes for 14 days. The CD19Bi armed murine ATC were tested in a ⁵¹Cr cytotoxicity assay using A20 lymphoma cell targets (FIG. 41). These studies show that armed ATC can be produced.

### Example 26

### Cord Blood Mononuclear Cells (CBMC) and CBATC Do not Respond to Each Other

In order to determine whether dual cord transplants would be feasible, applicants tested CB for alloreactivity to normal allogeneic peripheral blood mononuclear cells (PBMC) and tested CBATC from one CB for alloreactivity to cord blood mononuclear cells from a second cord blood (CB2). The data in FIG. 6 (representative of 3 experiments) show that CB1 or CB1ATC do not react to irradiated PBMC or irradiated CB2. It should be noted that the CBs used for this study were not HLA-typed.

In summary, CBATC can be consistently expanded in IL-2 from cryopreserved CB units nearly two logs in 2 weeks. Consistent with applicants' findings using peripheral blood, CBATC can be armed with Her2B1 or CD20Bi to target Her2+ and CD20+ targets. Dose titrations show that the optimal arming dose range is approximately 50 ng for each BiAb/10⁶ ATC. Specific cytotoxicity as measured by ⁵¹Cr release assays or by IFNγ Elispots responses peaked between 8 and 12 days for both HER2B1 and CD20Bi armed ATC. These studies show that functional, specific, and cytotoxic T cells can be generated from fresh or frozen CB and may be used to target tumors cells in vivo. The MLC data strongly support the use of CBATC derived from a second CB unit to help the primary CB unit engraft since there is little alloreactivity in whole CB, CBATC, or armed CBATC. As predicted, CB, CBATC, or armed CBATC do not react to each other.

### Example 27

### MNC do not React to Normal Donor (ND) in MLR as Strong as ND React to Each Other

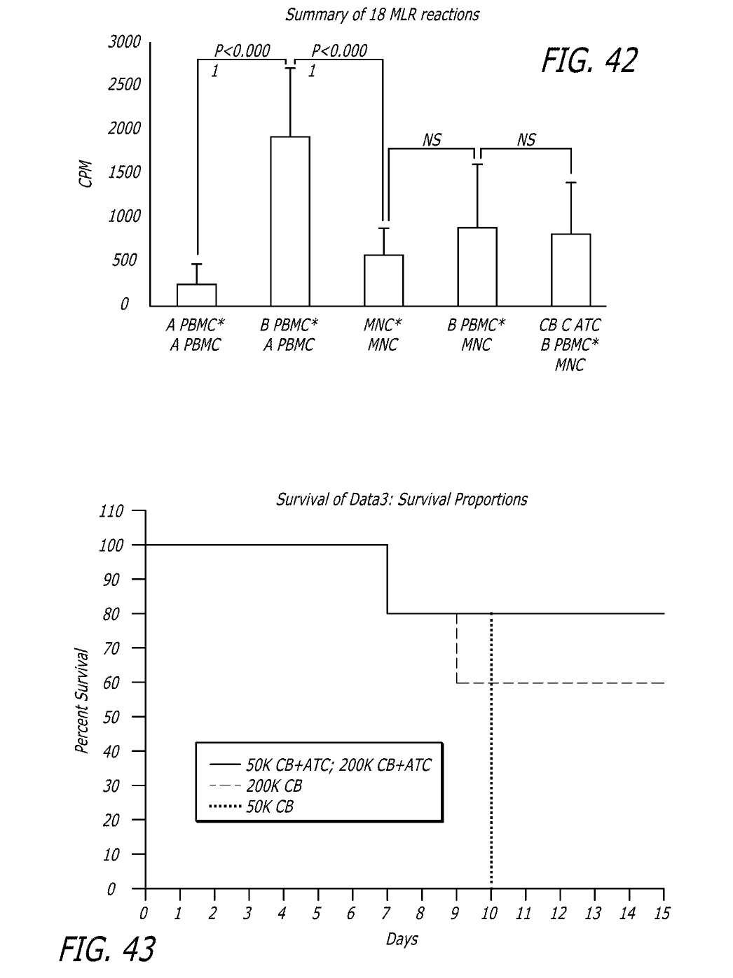

In order to determine whether cord blood mononuclear cells would respond in mixed lymphocyte culture reactions and whether anti-CD3 activated T cells (ATC derived from cord blood) would respond to alloantigens, applicants tested 18 separate cord blood samples. FIG. 42 shows that cord blood (CB) mononuclear cells did not react to Normal Donor (ND) PBMC in the MLR as strong as normal donors reacted to each other (tt=0.001). While average response of ND PBMC to allo ND PBMC was 8.1 fold (SD 3.2 fold) vs the mixed lymphocyte reaction to autologous ND PBMC (tt=0.00002, N=8), CB mononuclear cells proliferated only 1.6 fold (std 1.3 fold) over the reaction to auto CB mononuclear cells (tt=0.04, N=18) in response to allogeneic ND PBMC. Addition of auto CB ATC to the allo reaction to ND PBMC did not influence the degree of the allo response and was equal to 1.5 fold (std 1.1) over the reaction to auto-CB mononuclear cells and 0.9 fold (std 0.6) over the allo reaction without CB ATC added to the MLR.

### Example 28

### ATC Enhances Engraftment of Umbilical Cord Blood Hematopoietic Stem Cells (UCBHSC) in SCID/Biege Mice

Female, specified pathogen-free SCID/Biege mice 8 to 10 weeks of age were irradiated using 3.5 Gy TBI, delivered by a ¹³⁷Cs source adapted for the irradiation of mice, 24 hours before transplantation of umbilical cord blood hematopoietic stem cells (UCBHSC). Conditioned SCID/Biege mice were engrafted with various doses and combination of cells (Table 1) by IV injection into a lateral tail vein. As shown in FIG. 43, mice were monitored on daily basis for the survival and to determine whether combination of UCBHSC with activated T-cells (ATC) can rescue these mice by enhancing the engraftment of UCBHSC. Applicants' data suggest that ATC can enhance engraftment when given with very low numbers of UCBHSC.

TABLE 1
Determination of dose of UCBHSC alone or in combination with cord
blood ATC.

| Groups | Cell dose |
|---|---|
| 1 | 50,000 UCBHSC only |
| 2 | 200,000 UCBHSC only |
| 3 | 50,000 UCBHSC + 0.5 × 10\hat{ }6 ATC |
| 4 | 200,000 UCBHSC + 0.5 × 10\hat{ }6 ATC |
| 5 | 50,000 UCBHSC + 0.5 × 10\hat{ }6 aATC* |

*ATCs armed with CD45 × E-selectin bispecific antibodies

### Example 29

### Evaluation of the Effect of CB T Cell or CB ATC Function from a Second CB (CB2) on the Engraftment of the First CB (CB1) in a Nod-SCIDIL2RECEPTORγCHAIN^{NULL}(NSIγ^{NU&LL}) Mouse Model

Applicants will 1) confirm, optimize, and characterize the conditions for immune and myeloid engraftment; 2) determine the effects of adding unfractionated cells, purified T cells, ATC, and armed ATC from CD2 on engraftment CB1; and 3) determine the CBATC subset(s) from CB2 that enhances or modulates engraftment of CB1. If T cells, ATC, or armed ATC can enhance or facilitate hematopoietic engraftment and accelerate lymphoid reconstitution after cord blood transplant, a significantly greater number of patients will have donors for unrelated cord blood transplant. It is important to define the qualitative and quantitative contribution of T cells, ATC, and armed ATC on engraftment in a human-NSIγ^{null} model before performing phase I clinical trials using ATC or armed ATC to enhance engraftment after CBSCT. Engraftment has consistently achieved with 1×10⁵ or 2×10⁴ CD34+(CD38−)⁵⁷ Immune reconstitutions studies after CB show a delay in both hematopoietic and lymphopoietic reconstitution and these effects are attributed to cell dose of HSC and the impaired immune status of the newborn immune system (immunologically naïve). The addition of ATC may provide critical components to the HSC growth environment and/or provide vital growth factors for the maturation of HSC and/or accelerate immune development. The terms “Immune reconstitution” would not be the appropriate term since the transplanted CB immune system is immunologically naïve. Therefore, based on prior reports using the NSIγ^{null} model using CB for the development of a functional immune and hematological reconstitution for CBSCT and reports using dual CB transplants, Applicants will establish the NSIγ^{null} model for human CB engraftment and expansion and test the hypothesis that CBATC or armed CBATC would enhance engraftment.

To conduct the experiments suggested above the following materials and methods will be employed. The initial strategic experiments will be performed to define a dose response curve for engraftment of a single CB with decreasing doses of CB derived CD34+ cells. Since it has been well-defined that 1×10⁵ CD34+ cell could engraft the mice consistently, Applicants will use this dose as the starting dose and perform a dose reduction titration to find the dose-limiting dose for engraftment. All mice will be given 3.5 Gy of TBI to ablate their bone marrows. There will be 5-10 mice per group, each group will receive: 1) no cells; 2) 1×10⁵; 3) 5×10⁴; 4) 2.0×10⁴; and 5) 1.0×10⁴ CD34+ positively selected cells using Mitlenyl beads to define a range in which roughly 25% of the mice engraft with hematopoietic and lymphopoietic lineages. CD34 cell dose from a single CB required to rescue ˜25% of the NSIγ^{null} mice, the rescue dose of CD34+ cells is defined as RDCD34. The experiment will be repeated with lower doses of CD34+ cells if the first dose titration does not define a RDCD34. Survival for 30 days will be considered successful engraftment if the mouse has an ANC of 500 cells/mm³. Phenotyping of the spleen and bone marrow will be done on mice that survive 30 days for hematopoietic markers (CD34, CD33) and lymphopoietic markers (CD3, CD4, CD8, CD20, CD19, CD56). A Chi-Square analysis will be used to analyze survival since 30 days will be taken as a successful endpoint⁶.

After establishing the RDCD34 for CB1, the RDCD34 from CB1 will be held constant and a series of experiments will be performed using whole unfractionated CB, T cells, T cell subsets, activated T cells, activated T cell subsets, armed ATC, armed ATC subsets from CB1. However, the first experiment will use the excess of autologous whole unfractionated CB, CBATC, and CB armed ATC with unfractionated CB and purified T cells from CB1 as controls (10 mice per group with 2 replicates). Since the markedly enhancing doses of ASC was 1×10⁶ in applicants' mouse model, Applicants selected as a first order estimate 2×10⁶ of autologous unfractionated CB (control), CB T cells (control), activated T cells, or armed ATC for the add back to the RDCD34 to clearly show that there is an engraftment enhancing effect as measured by survival and cell counts in the blood, spleen, and bone marrow. The autologous CBATC and armed ATC will be expanded for 6-14 days prior to the co-injection experiments. If the 2×10⁶ dose of these populations are sufficient to markedly enhance engraftment in a specific group, a dose titration will focus on autologous ATC, and armed ATC to more clearly define the effect of adding that specific population to the RDCD34. A total of 10 mice will be transplanted in each group. The dose titrations will be done with roughly ½ log decreases in the study population.

All of the NSIγ^{null} mice will receive the same RDCD34. The equal doses of whole CB, ATC and armed ATC derived from CB1 will be added to RDCD34 from CB1. This set would provide the autologous control for these experiments. Whole CB, ATC or armed ATC (2×10⁶ or 5×10⁶) from CB1 will be added to the RDCD34 from CB1 and whole CB, ATC or armed ATC (2×10⁶ or 5×10⁶) CB2 will be added to RDCD34 from CB1. Eight-ten mice will be treated in each group. At 30 days after transplant, the mice will be euthanized and blood will be drawn for absolute neutrophil and lymphocyte counts and the spleen and bone marrow cell harvested and counted after 30 days.

In order to determine whether adding ATC or armed ATC from a second unrelated CB2 would enhance engraftment of the CD34+ cells from CB1, Applicants have designed the experiment will be performed to evaluate engraftment in the following groups: 1) NSIγ^{null} mice co-injected with RDCD34+ from CB1 and whole CB2 (two different doses); 2) NSIγ^{null} mice co-injected with RDCD34+ from CB1 and ATC or armed ATC from CB1 (two doses); 3) NSIγ^{null} mice co-injected with RDCD34+ from CB1 and ATC or armed ATC from CB2 (two doses); 4) NSIγ^{null} mice co-injected with RDCD34+ from CB1 and whole CB2 (one dose); 5) NSIγ^{null} mice co-injected with RDCD34+ from CB1 and armed CD4 or CD8ATC from CB1 (two doses); 6) NSIγ^{null} mice co-injected with RDCD34+ from CB1 and whole CB2 (one dose).

If there is no difference between unarmed and armed ATC, all subsequent experiments will be done using only armed ATC since they would be the “product” that will be used for the clinical trial. Assuming that the experiments involving the addition of ATC or armed ATC from CB2 show promise for enhancing engraftment, Applicants will then focus on armed CD4+CBATC and CD8+ CBATC from CB2 to be tested in the same system. Again Applicants would use whole CB1 and whole CB2 as controls. Each population will be co-transplanted with CB1 to determine the effect of each of the population with 8-10 mice for each group with 2-3 replicates. The ATC subset and armed ATC subset that provides the highest levels of faciltatory activity for engraftment will then be dose titration to define the dose-response curve.

By using a well-established model for measuring the functional development of human hematopoietic and lymphopoietic systems, applicants anticipate that the DSIγ^{null} mouse model can be developed without significant problems since Applicants already have experience working with immunodeficient mice. The mice are commercially available. All of the techniques and procedures for growing, harvesting, and arming ATC as well as purifying CD34+ cells using Miltenyl beads are straightforward. The procedures for obtaining blood, spleen, and bone marrow are all in place. The procedures, reagents, and assays for arming any of the populations used for these experiments are all available. It is likely that ATC or armed ATC from one cord will provide help for hematopoiesis and lymphopoiesis of a second cord for the following reasons: 1) T cell depletion decreases engraftment; conversely, T cells provides helper activity for engraftment; 2) the addition of ASC to limiting doses of HSC markedly increased engraftment in a murine model¹⁸; 3) double CB transplants improve engraftment in NSI^{null}mice⁵⁶; 4) clinical results from double CB transplant show that patients can be successfully engrafted⁵⁸.

### REFERENCE LIST

1. Sen M, Wankowski D M, Garlie NK et al. Use of anti-CD3×anti-HER2/neu bispecific antibody for redirecting cytotoxicity of activated T cells toward HER2/neu Tumors. Journal of Hematotherapy & Stem Cell Research 2001; 10:247-260.
2. Lum H E, Miller M, Davol P A et al. Preclinical studies comparing different bispecific antibodies for redirecting T cell cytotoxicity to extracellular antigens on prostate carcinomas. Anticancer Res. 2005; 25:43-52.
3. Chan J K, Hamilton C A, Cheung M K et al. Enhanced killing of primary ovarian cancer by retargeting autologous cytokine-induced killer cells with bispecific antibodies: a preclinical study. Clin. Cancer Res. 2006; 12:1859-1867.
4. Reusch U, Sundaram M, Davol P A et al. Anti-CD3×anti-EGFR Bispecific Antibody Redirects T Cell Cytolytic Activity to EGFR-Positive Cancers In Vitro and in an Animal Model. Clin. Cancer Res. 2006; 12:183-190.
5. Gall J M, Grabert R C, Deaver M, Davol P A, Lum L G. Targeting CD20+ rituxan resistant B-cells with anti-CD3 activated T cells (ATC) armed with anti-CD3×anti-CD20 (CD20BI). [abstract]. Exp. Hematol. 2004; 32:35.
6. Jin N-R, Lum L G, Ratanatharathorn V, Sensenbrenner L L. Anti-CD3-activated splenocytes enhance survival in lethally irradiated mice after transplant of syngeneic hematopoietic stem cells. Exp. Hematol. 1995; 23:1331.
7. Brunstein C G, Baker K S, Wagner J E. Umbilical cord blood transplantation for myeloid malignancies. Curr. Opin. Hematol. 2007; 14:162-169.
8. Schoemans H, Theunissen K, Maertens J et al. Adult umbilical cord blood transplantation: a comprehensive review. Bone Marrow Transplant. 2006; 38:83-93.
9. Brunstein C G, Setubal D C, Wagner J E. Expanding the role of umbilical cord blood transplantation. Br. J. Haematol. 2007; 137:20-35.
10. Hofineister C C, Zhang J, Knight K L, Le P, Stiff P J. Ex vivo expansion of umbilical cord blood stem cells for transplantation: growing knowledge from the hematopoietic niche. Bone Marrow Transplant. 2007; 39:11-23.
11. Cornetta K, Laughlin M, Carter S et al. Umbilical cord blood transplantation in adults: results of the prospective Cord Blood Transplantation (COBLT). Biol. Blood Marrow Transplant. 2005; 11:149-160.
12. Komanduri K V, St John L S, de L M et al. Delayed immune reconstitution after cord blood transplantation is characterized by impaired thymopoiesis and late memory T cell skewing. Blood 2007
13. Majhail N S, Brunstein C G, Wagner J E. Double umbilical cord blood transplantation. Curr. Opin. Immunol. 2006; 18:571-575.
14. Rocha V, Gluckman E. Clinical use of umbilical cord blood hematopoietic stem cells. Biol. Blood Marrow Transplant. 2006; 12:34-41.
15. Hofineister C C, Zhang J, Knight K L, Le P, Stiff P J. Ex vivo expansion of umbilical cord blood stem cells for transplantation: growing knowledge from the hematopoietic niche. Bone Marrow Transplant. 2007; 39:11-23.
16. Terakura S, Azuma E, Murata M et al. Hematopoietic engraftment in recipients of unrelated donor umbilical cord blood is affected by the CD34+ and CD8+ cell doses. Biol. Blood Marrow Transplant. 2007; 13:822-830.
17. Magro E, Regidor C, Cabrera R et al. Early hematopoietic recovery after single unit unrelated cord blood transplantation in adults supported by co-infusion of mobilized stem cells from a third party donor. Haematologica 2006; 91:640-648.
18. Jin N R, Lum L G, Ratanatharathorn V, Sensenbrenner L L. Anti-CD3-activated splenocytes enhance survival in lethally irradiated mice after transplant of syngeneic hematopoietic stem cells. Exp. Hematol. 1995; 23:1331-1336.
19. Hexner E O, net-Desnoyers G A, Zhang Yet al. Umbilical cord blood xenografts in immunodeficient mice reveal that T cells enhance hematopoietic engraftment beyond overcoming immune barriers by stimulating stem cell differentiation. Biol. Blood Marrow Transplant. 2007; 13:1135-1144.
20. Horowitz M M, Gale R P, Sondel P M et al. Graft-versus-leukemia reactions after bone marrow transplantation. Blood 1990; 75:555-562.
21. Kolb H J, Holler E. Adoptive immunotherapy with donor lymphocyte transfusions [Review]. Curr. Opin. Oncol. 1997; 9:139-145.
22. Uberti J P, Joshi I, Ueda M et al. Preclinical studies using immobilized OKT3 to activate human T cells for adoptive immunotherapy: optimal conditions for the proliferation and induction of non-MHC restricted cytotoxicity. Clin. Immunol. Immunopathol. 1994; 70:234-240.
23. Chen B P, Malkovsky M, Hank J A, Sondel P M. Nonrestricted cytotoxicity mediated by interleukin 2-expanded leukocytes is inhibited by anti-LFA-1 monoclonal antibodies (MoAb) but potentiated by anti-CD3 MoAB. Cell Immunol. 1987; 110:282-293.
24. Ochoa A C, Gromo G, Alter B J, Sondel P M, Bach F H. Long-term growth of lymphokine-activated killer (LAK) cell: role of anti-CD3, beta-IL 1, interferon-gamma and -beta. J. Immunol. 1987; 138:2728-2733.
25. Anderson P M, Bach F H, Ochoa A C. Augmentation of cell number and LAK activity in peripheral blood mononuclear cells activated with anti-CD3 and interleukin-2. Preliminary results in children with acute lymphocytic leukemia and neuroblastoma. Cancer Immunol. Immunother. 1988; 27:82-88.
26. Yang S C, Fry K D, Grimm E A, Roth J A. Successful combination immunotherapy for the generation in vivo of antitumor activity with anti-CD3, interleukin 2, and tumor necrosis factor alpha. Arch. Surg. 1990; 125:220-225.
27. Ueda M, Joshi I D, Dan M et al. Preclinical studies for adoptive immunotherapy in bone marrow transplantation: II. Generation of anti-CD3 activated cytotoxic T cells from normal donors and autologous bone marrow transplant candidates. Transplantation 1993; 56:351-356.
28. Anderson P M, Blazar B R, Bach F H, Ochoa A C. Anti-CD3+IL-2-stimulated murine killer cells: in vitro generation and in vivo antitumor activity. J. Immunol. 1989; 142:1383-1394.
29. Anderson P M, Ochoa A C, Ramsay N K C, Hasz D, Weisdorf D. Anti-CD3+ interleukin-2 stimulation of marrow and blood: comparison of proliferation and cytotoxicity. Blood 1992; 80:1846-8153.
30. Ting C-C, Hargrove M E, Yun Y S. Augmentation by anti-T3 antibody of the lymphokine-activated killer cell-mediated cytotoxicity. J. Immunol. 1988; 141:741-748.
31. Ochoa A C, Hasz D E, Rezonzew R, Anderson P M, Bach F H. Lymphokine-activated killer activity in long-term cultures with anti-CD3 plus interleukin 2: identification and isolation of effector subsets. Cancer Res. 1989; 49:963-968.
32. Lum L G. Immunotherapy with Activated T Cells after High Dose Chemotherapy and PBSCT for Breast Cancer. In: Dicke K A, Keating A, eds. Charlottesville, N.Y.: Carden Jennings; 2000:95-105.
33. Davol P A, Smith J A, Kouttab N, Elfenbein G J, Lum L G. Anti-CD3×anti-HER2 bispecific antibody effectively redirects armed T cells to inhibit tumor development and growth in hormone-refractory prostate cancer-bearing severe combined immunodeficient beige mice. Clin Prostate Cancer 2004; 3:112-121.
34. Chan J K, Hamilton C A, Cheung M K et al. Enhanced killing of primary ovarian cancer by retargeting autologous cytokine-induced killer cells with bispecific antibodies: a preclinical study. Clin Cancer Res. 2006; 12:1859-1867.
35. Tita-Nwa F, Moldenhauer G, Herbst M et al. Cytokine-induced killer cells targeted by the novel bispecific antibody CD19×CD5 (HD37×T5.16) efficiently lyse B-lymphoma cells. Cancer Immunol. Immunother. 2007
36. McLaughlin P, Grillo-López A J, Link B K et al. Rituximab chimeric anti-CD20 monoclonal antibody therapy for relapsed indolent lymphoma: half of patients respond to a four-dose treatment program. J. Clin. Oncol. 1998; 16:2825-2833.
37. Foran J M, Gupta R K, Cunningham D et al. A UK multicentre phase II study of rituximab (chimaeric anti-CD20 monoclonal antibody) in patients with follicular lymphoma, with PCR monitoring of molecular response. Br. J. Haematol. 2000; 109:81-88.
38. Colombat P, Salles G, Brousse N et al. Rituximab (anti-CD20 monoclonal antibody) as single first-line therapy for patients with follicular lymphoma with a low tumor burden: clinical and molecular evaluation. Blood 2001; 97:101-106.
39. Hainsworth J D, Burris H A, III, Morrissey L H et al. Rituximab monoclonal antibody as initial systemic therapy for patients with low-grade non-Hodgkin lymphoma. Blood 2000; 95:3052-3056.
40. Gall J M, Davol P A, Grabert R C, Deaver M, Lum L G. T cells armed with anti-CD3× anti-CD20 bispecific antibody enhance killing of CD20+ malignant B cells and bypass complement-mediated rituximab resistance in vitro. Exp. Hematol. 2005; 33:452-459.
41. Gall J M, Davol P A, Grabert R C, Deaver M, Lum L G. T cells armed with anti-CD3× anti-CD20 bispecific antibody enhance killing of CD20+ malignant B cells and bypass complement-mediated rituximab resistance in vitro. Exp. Hematol. 2005; 33:452-459.
42. Wrzesinski C, Paulos C M, Gattinoni L et al. Hematopoietic stem cells promote the expansion and function of adoptively transferred antitumor CD8 T cells. J. Clin Invest 2007; 117:492-501.
43. Rapoport A P, Stadtmauer E A, Aqui N et al. Restoration of immunity in lymphopenic individuals with cancer by vaccination and adoptive T-cell transfer. Nat. Med. 2005; 11:1230-1237.
44. Wei Y M, Cao Q, Zhou H Y et al. Ex vivo expansion of T, NK and CD34+ cells from umbilical cord blood. Zhongguo Shi Yan.Xue.Ye.Xue.Za Zhi. 2005; 13:1076-1081.
45. Kobari L, Giarratana M C, Gluckman J C, Douay L, Rosenzwajg M. Ex vivo expansion does not alter the capacity of umbilical cord blood CD34+ cells to generate functional T lymphocytes and dendritic cells. Stem Cells 2006; 24:2150-2157.
46. Serrano L M, Pfeiffer T, Olivares S et al. Differentiation of naive cord-blood T cells into CD19-specific cytolytic effectors for posttransplantation adoptive immunotherapy. Blood 2006; 107:2643-2652.
47. Singh H, Serrano L M, Pfeiffer T et al. Combining adoptive cellular and immunocytokine therapies to improve treatment of B-lineage malignancy. Cancer Res. 2007; 67:2872-2880.
48. Lum L G, Davol P, Grabert R et al. Targeting Pancreatic Cancer with Armed Activated T Cells Directed at Her2/neu Receptors. [abstract]. Exp. Hematol. 2002;
49. Davol P A, Smith J A, Kouttab N, Elfenbein G J, Lum L G. Anti-CD3×Anti-HER2 bispecific antibody effectively redirects armed T cells to inhibit tumor development and growth in hormone-refractory prostate cancer-bearing SCID-Beige mice. Clin. Prostate Cancer 2004; 3:112-121.
50. Grabert R C, Cousens L P, Smith J A et al. Human T cells armed with Her2/neu bispecific antibodies divide, are cytotoxic, and secrete cytokines with repeated stimulation. Clin. Cancer Res. 2006; 12:569-576.
51. Treon S P, Mitsiades C, Mitsiades N et al. Tumor cell expression of CD59 is associated with resistance to CD20 serotherapy in patients with B-cell malignancies. J. Immunother. 2001; 24:263-271.
52. Gall J M, Davol P A, Grabert R C, Deaver M, Lum L G. T cells armed with anti-CD3× anti-CD20 bispecific antibody enhance killing of CD20+ malignant B-cells and bypass complement-mediated Rituximab-resistance in vitro. Exp. Hematol. 2005; 33:452-459.
53. Wilson E G. Probable inference, the law of succession and statistical inference. Journal of the Statistical Association 2007; 22:209-212.
54. Klein J P and Moeschberger M L. Survival Analysis, 2nd ed. 2003. New York, Springer-Verlag.
55. Hintze J. PASS 2002. Kaysville Utah: Number Crunching Statistical Systems. 2002.
56. Nauta A J, Kruisselbrink A B, Lurvink E et al. Enhanced engraftment of umbilical cord blood-derived stem cells in NOD/SCID mice by cotransplantation of a second unrelated cord blood unit. Exp. Hematol. 2005; 33:1249-1256.
57. Ishikawa F, Yasukawa M, Lyons B et al. Development of functional human blood and immune systems in NOD/SCID/IL2 receptor {gamma} chain (null) mice. Blood 2005; 106:1565-1573.
58. Jaing T H, Yang C P, Hung I J et al. Transplantation of unrelated donor umbilical cord blood utilizing double-unit grafts for five teenagers with transfusion-dependent thalassemia. Bone Marrow Transplant. 2007; 40:307-311.
59. Gluckman E, Rocha V. Donor selection for unrelated cord blood transplants. Curr. Opin. Immunol. 2006; 18:565-570.

All patents, patent applications, provisional applications, and publications referred to or cited herein are incorporated by reference in their entirety, including all figures and tables, to the extent they are not inconsistent with the explicit teachings of this specification.

1. A composition comprising cord blood mononuclear cells wherein said cord mononuclear cells do not react to allo-antigens on other cord bloods.

2. A composition comprising cord blood T cells and other mononuclear cell components in cord blood wherein said cord blood T cells and other mononuclear cell components in cord blood do not react to allo-antigens in adult peripheral blood on other cord bloods.

3. A composition comprising anti-CD3 activated cord blood T cells and other activated mononuclear cell components in cord blood wherein said anti-CD3 activated cord blood T cells and other activated mononuclear cell components in cord blood do not react to allo-antigens on other cord bloods.

4-11. (canceled)
